(19) United States
(12) Patent Application Publication
(10) Pub. No.: US 2010/0068093 A1
(43) Pub. Date: Mar. 18, 2010

(54) Identification of a Strip Type by the Meter Using Conductive Patterns on the Strip

(75) Inventors: Yi Wang, San Ramon, CA (US); Shridhara Alva Karinka, Pleasanton, CA (US); John R. Galasso, Danville, CA (US)

Correspondence Address: Abbott Diabetes Care Inc.;Bozicevic, Field & Francis LLP, 1900 University Ave, Suite 200, East Palo Alto, CA, 94303, US

(73) Assignee: Abbot Diabetes Care Inc., Alameda, CA (US)

(21) Appl. No.: 12/622,179

(22) Filed: Nov. 19, 2009

Related U.S. Application Data
(63) Continuation of application No. 12/463,223, filed on May 8, 2009.
Application No. 12/463,223 is a continuation of application No. 12/110,240, filed on Apr. 25, 2008.
(60) Provisional application No. 60/914,650, filed on Apr. 27, 2007.

(51) Int. Cl. G01N 33/50; G01N 31/22

(52) U.S. Cl. 422/56; 422/68.1

(57) ABSTRACT

A test strip including a reagent end for receiving a body fluid sample and an insertion end which is received at a test strip receptacle slot of a measuring device, the insertion end including one or more on-strip indicators for indicating to a measuring device that the test strip has been inserted into the device so that the device will power-up in preparation to perform a test, and the one or more on-strip indicators having a distinct pattern indicating whether calibration is automatic or whether the user needs to input calibration information to the said device, and corresponding methods are disclosed.

### RELATED APPLICATION

The present application claims priority under 35 U.S.C. §119(e) to U.S. provisional application No. 60/914,650 filed Apr. 27, 2007, entitled “Identification of a Strip Type by the Meter Using Conductive Patterns on the Strip”, the disclosure of which is incorporated herein by reference for all purposes.

### BACKGROUND

Biosensors, also referred to as analytical sensors or merely sensors, often in the form of test strips, are commonly used in chemistry and medicine to determine the presence and concentration of a biological analytes in samples, such as body fluid samples. Such biosensors are used, for example, to monitor blood glucose levels in diabetic patients and lactate during critical care events. As sensors continue to be used, there continues to be an interest in sensors that are easy to manufacture and easy for a patient to use.

Sensors are typically used in conjunction with meters which have slots for receiving the test strips, and analytical components for determine an amount of analyte in a sample applied to the strip. It is desired to have a user-friendly way for a user and/or a meter to determine what kind of strip is being used. Strips or packages of strips typically have calibration information associated with them. There are different calibration procedures available, such as specific calibration testing strips with memory chips and calibration codes on boxes of strips for manual entry to the meter by a user. It is desired to also have strip-meter systems that autocalibrate with calibration information stored on the strip or is pre-calibrated to the strip to be used with the meter, i.e., without any need for input or action by a user. As a result, it is also desired to have a way to indicate to the user and/or to the meter what type of calibration is associated with certain strips or groups of strips.

### BRIEF DESCRIPTION OF THE DRAWINGS

Referring now to the drawings, wherein like reference numerals and letters indicate corresponding structure throughout the several views:

FIG. 1 is a schematic view of a first embodiment of a sensor strip in accordance with the present invention;

FIG. 2A is an exploded view of the sensor strip shown in FIG. 1, the layers illustrated individually with the electrodes in a first configuration;

FIG. 2B is a top view of the sensor strip shown in FIGS. 1 and 2A;

FIG. 3A is a schematic view of a second embodiment of a sensor strip in accordance with the present invention, the layer illustrated individually with the electrodes in a second configuration;

FIG. 3B is a top view of the sensor strip shown in FIG. 3A;

FIG. 4 is a top view of the first substrate of the sensor strip of FIGS. 3A and 3B;

FIG. 5A is a top view of a first example configuration for a suitable insertion monitor in accordance with the present invention;

FIG. 5B is a top view of a second example configuration for a suitable insertion monitor in accordance with the present invention;

FIG. 5C is a top view of a third example configuration for a suitable insertion monitor in accordance with the present invention;

FIG. 5D is a top view of a fourth example configuration for a suitable insertion monitor in accordance with the present invention;

FIG. 6A illustrates a top view of one embodiment of a sheet of sensor components, according to the invention;

FIG. 6B illustrates a top view of another embodiment of a sheet of sensor components, according to the invention;

FIG. 7A is a top perspective view of a sensor strip positioned for insertion within an electrical connector device in accordance with the present invention;

FIG. 7B is an exploded view of the electrical connector device of FIG. 7A;

FIG. 8 is a schematic view of another embodiment of a sensor strip;

FIG. 9 is an exploded view of the sensor strip shown in FIG. 8, the layers illustrated individually with the electrodes in a first configuration;

FIG. 10 shows another embodiment of a test strip sensor;

FIG. 11 shows another embodiment of a test strip sensor;

FIGS. 12A-12F shows embodiments of test strip sensors that have various wakeup bar shapes and various contours of their reagent ends or sample application ends;

FIGS. 13A-13E illustrate various layers of an embodiment of a test strip having a distinctly contoured reagent end and wake-up bar shape;

FIGS. 14A-14E illustrate various layers of the reagent end of the test strip illustrated at FIGS. 13A-13E; and

FIGS. 15A-15E illustrate embodiments of strips having distinct protrusions on opposing sides.

FIGS. 16A-16D illustrate embodiments of strips having a distinct protrusion at one end.

### DETAILED DESCRIPTION

Blood glucose monitoring systems use an electronic device (meter) and a disposable test strip. The test strip can be electrochemical or photometric.

These test strips contain chemistry that reacts with an analyte (such as glucose) in a sample, such as a body fluid sample, such that a meter can read a signal proportional to the concentration of the analyte. Test strips are inserted into the meter for performing a test. The meter recognizes the insertion of a test strip and then upon application of the sample performs the measurement.

The recognition of the strip can be done mechanically. When a strip is inserted, the strip would open or close electronic circuits in the meter indicating the insertion of a strip. Only test strips that meet the physical requirements will activate the meter.

The recognition of the strip can also be done electronically. In this case, the meter detects an electronic signal (such as electrical continuity) from the test strip and activates the meter for performing the assay. The electronic signal can be in the form of a conducting electrode or combination of electrodes. FreeStyle test strips from Abbott Diabetes Care use a conducting stripe (wake-up bar) to activate the meter upon insertion of the strip.

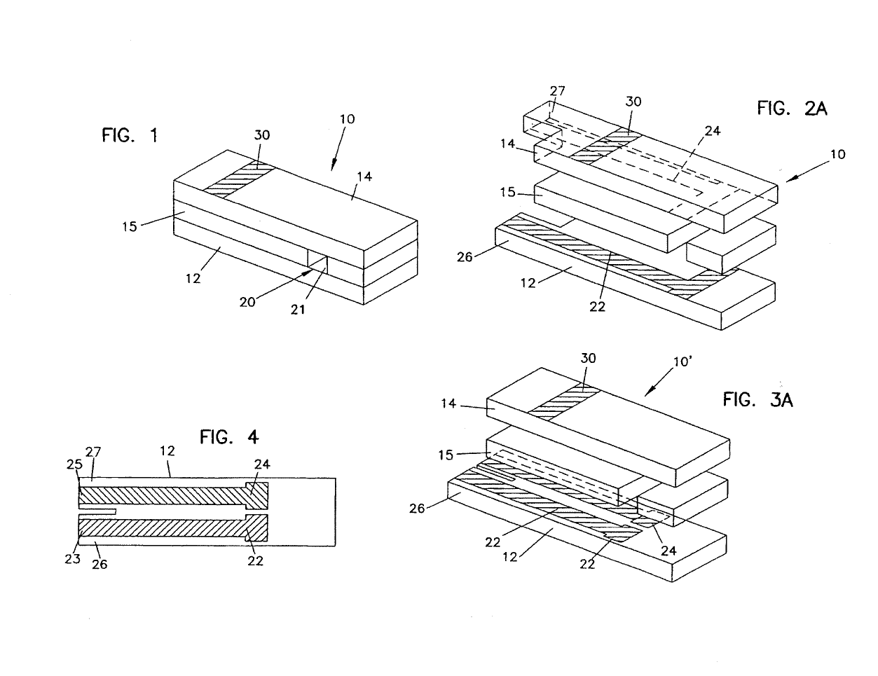

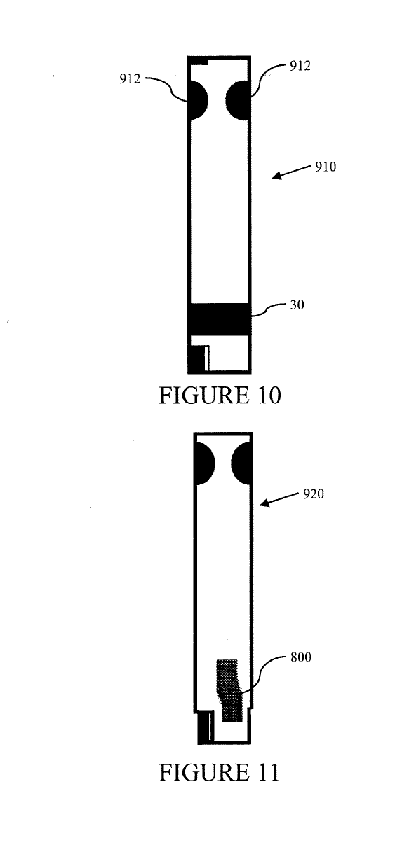

Embodiments for improvements in the mechanical and/or electronic detection of strips are provided herein. In certain embodiments, a wake-up bar can be patterned to differentiate strips that require, for example, different calibration information. FIGS. 1, 2B, 3B, 5A-5D, 7A-7B, 8-11, 12A-12F, and 13A illustrates different configurations of wake-up bars that may be distinguished by a user and/or by a meter, where such are exemplary configurations and are not intended to limit the configurations that may be utilized. As an exemplary illustration, the pattern of the wake-up bar 30 shown in FIG. 10 may be used for electrochemical test strips, e.g., FreeStyle® blood glucose monitoring strips. This rectangular wake-up bar 30 may indicate to the meter or user that the strip requires the user to input the calibration information to the meter before performing the test. In contrast, FIG. 11 shows a wake-up bar 800 having a different pattern than the wake-up bar 30 of FIG. 10. The pattern of wake-up bar 800 may indicate information to the meter and/or user, e.g., may indicate to the meter or user that the user does not have to input the calibration information to the meter, i.e., calibration is automatic with these types of strips. This may reduce errors that may otherwise be caused by a user miscoding the meter, or the meter miscoding itself.

A single meter can be configured to accept both these kind of strips or to accept only one kind of strip. In other words, the wake-up bar design can make the strips mutually exclusive, eliminating the cross use. Different mechanical configurations of strips are illustrated throughout FIGS. 1-16D, and specifically FIGS. 8-9, 12D-12F, 13A-14E, and 15A-16D illustrates embodiments of strips that differ mechanically from typical, substantially rectangular-shaped strips such as illustrated at FIGS. 10-11 and 12A-12C.

Different wake-up bar designs or strip shapes can be used which permit a user and/or a meter to distinguish whether calibration is automatic or whether a user must perform some act such as placing a calibration strip into a meter or entering calibration information to the meter.

The different wake-up bar designs or strip shapes may also be used to distinguish assay algorithms. For example, one assay algorithm may determine a current peak, and then determine how long it takes for the current to reduce to 50% of the peak value, while another algorithm may determine how long it takes for the current to reduce to 75%, or 25%, etc. A meter reading a test strip may identify the particular pattern and implement the appropriate assay algorithm. In some embodiments, a meter may store a plurality of different assay algorithms from which to implement, or may store only one, e.g., identification of a wake up bar pattern would confirm that the meter implement the stored algorithm.

The different wake-up bar designs or strip shapes may also be used to distinguish strips of different sizes. For example, a smaller strip will generally yield smaller currents, and so it may be desired to turn a gain up on a meter when the smaller strips are being used to measure an applied analyte, while the gain would be turned back down when a larger strip is inserted into the meter. The meter, or the user, may understand from the shape of the wake-up bar or strip end which gain amount to use and cause the relevant circuit in the meter to be adjusted.

The different wake-up bar designs or strip shapes may also be used to distinguish different hardware configurations of the meter and/or so that the meter and/or the user knows whether to apply a certain voltage such as 100 mV or 200 mV in order to produce a current within a desired range.

An existing or older meter may not provide adequate results when a new strip is inserted into it. Thus, it can be dangerous for a diabetic to user the old meter with new strips. A new strip is provided that when inserted into an old or existing meter will not turn the meter on. E.g., an electrode in the meter may not complete a circuit with the wake-up bar of the new strip even though it would do so with an older strip.

The wake-up bar can differ not only in shape, but also in conductivity and/or thickness and/or material composition, and these may be distinguished by the meter. For example, silver ink may be used in one strip, while carbon ink may be used in another strip, wherein the carbon ink may have 100× greater resistance than the silver ink for a same wake-up bar design, such as 500 ohms to 5 ohms. The meter could distinguish the signals it gets depending on which of these materials is used. In addition, a very thick bar would have lower resistance than a very thin bar of the same design shape looking at it from overhead.

In a first embodiment, a glucose monitoring test strip includes a reagent end for receiving a body fluid sample and an insertion end which is received at a glucose meter test strip receptacle slot. The insertion end includes one or more wake-up bars, such as may be conductive traces, wires or other conductive or semiconductive materials, for indicating to a glucose meter that the test strip has been inserted into the meter, e.g., by completing a circuit which then passes a threshold current or has a particular voltage drop for certain input current, so that the meter will power-up in preparing to perform a test. The one or more wake-up bars, or other conductive traces, wires, or other components, have a distinct pattern indicating whether calibration is automatic or whether the user needs to input certain calibration information to the meter.

In a second embodiment, the insertion end of a test strip includes a distinct contour, perimeter pattern, shape, thickness, width or other geometric, material or other measurable property, or combinations thereof, indicating whether calibration is automatic or whether the user needs to input certain calibration information to the meter, or other information.

In a third embodiment, the insertion end includes one or more wake-up bars for indicating to a glucose meter that the test strip has been inserted into the meter so that the meter will power-up in preparing to perform a test. The one or more wake-up bars have a distinct pattern indicating a particular calibration requirement corresponding to a type of the test strip which contrasts with different calibration requirements of at least one other type of test strip.

In a fourth embodiment, the insertion end includes a distinct contour, perimeter pattern, shape, thickness, width or other geometric, material or other measurable property, or combinations thereof, indicating a particular calibration requirement corresponding to a type of the test strip which contrasts with different calibration requirements of at least one other type of test strip.

Variations of these embodiment and those described below, e.g., of particular patterns to keep strips mutually exclusive can be envisioned. For example, a user may needs to enter a calibration code corresponding to a certain package of strips or type of strip or package of strips. A user may instead need to insert a special test strip that includes a memory chip that has calibration information on it for transmitting to the meter. The calibration may instead be automatic. As for the strips, they may have co-planar working and reference electrodes at the reagent end (on a same surface of the strip), or they may have opposing working and reference electrodes at the reagent end (on different surfaces of the strip). The strips may be top filled, side filled or corner filled. One end, e.g., the reagent end, may have a rectangular or tapered shape. The strips may include a sample fill area that may include one or more protrusions, cut-outs, notches, or indentations. The strip may be configured for amperometric or coulometric measurements.

### Sensors Generally

The sensors of various embodiments provide a method for the detection and quantification of an analyte. In general, a method and sensor are described herein for analysis of an analyte in a sample, e.g., a small volume sample, by, for example, coulometry, amperometry and/or potentiometry. The sensor also includes a sample chamber to hold the sample in electrolytic contact with the working electrode.

In certain embodiments, the working electrode faces a counter electrode, forming a sample chamber, between the two electrodes, that is sized to contain no more than about 1 μL of sample, e.g., no more than about 0.5 μL, e.g., no more than about 0.32 μL, e.g., no more than about 0.25 μL, e.g., no more than about 0.1 μL of sample.

In certain embodiments, the working electrode and the counter electrode are coplanar. A sample chamber is constructed over the working and counter electrode that is sized to contain no more than about 1 μL of sample, e.g., no more than about 0.5 μL, e.g., no more than about 0.32 μL, e.g., no more than about 0.25 μL, e.g., no more than about 0.1 μL of sample.

In certain embodiments, a sensor is configured for insertion into an electronic meter and is provided with a working electrode and a counter electrode, and a conductive insertion monitor which provides electrical contact with the electronic meter if the sensor is properly inserted into the meter. The conductive insertion monitor is configured and arranged to close an electrical circuit when the sensor is properly inserted into the electronic connector.

The sensors of certain embodiments can be configured for side-filling or tip-filling or top filling. In addition, in some embodiments, the sensor may be part of an integrated sample acquisition and analyte measurement device. The integrated sample acquisition and analyte measurement device can include the sensor and a skin piercing member, so that the device can be used to pierce the skin of a user to cause flow of a fluid sample, such as blood, that can then be collected by the sensor. In at least some embodiments, the fluid sample can be collected without moving the integrated sample acquisition and analyte measurement device.

In one embodiment, the sensor is connected with an electrical device, to provide a processor coupled to the sensor.

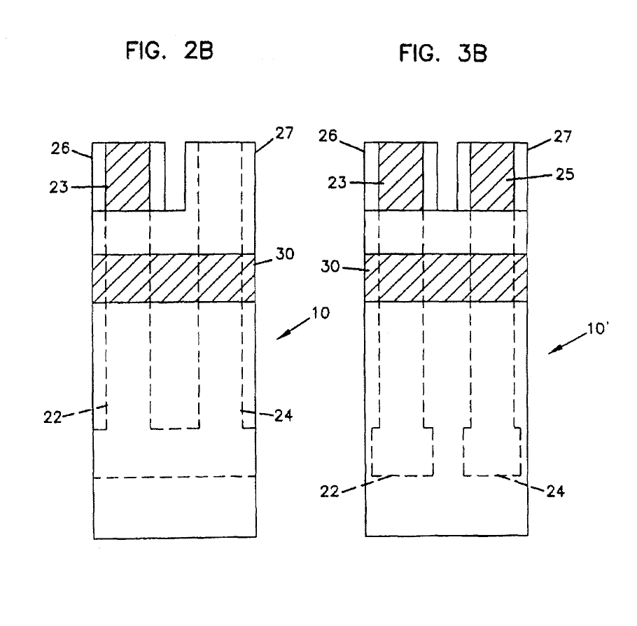

Referring to the Drawings in general and FIGS. 1 and 2A in particular, a first embodiment of a sensor strip 10 is schematically illustrated. Sensor strip 10 has a first substrate 12, a second substrate 14, and a spacer 15 positioned therebetween. Sensor strip 10 includes at least one working electrode 22 and at least one counter electrode 24. Sensor strip 10 also includes insertion monitor 30.

### Sensor Strips

Referring to FIGS. 1, 2A and 2B in particular, sensor strip 10 has first substrate 12, second substrate 14, and spacer 15 positioned therebetween. Sensor strip 10 includes working electrode 22, counter electrode 24 and insertion monitor 30. Sensor strip 10 is a layered construction, in certain embodiments having a generally rectangular shape, i.e., its length is longer than its width, although other shapes are possible as well. Sensor strip 10′ of FIGS. 3A, 3B and 4 also has first substrate 12, second substrate 14, spacer 15, working electrode 22, counter electrode 24 and insertion monitor 30.

The dimensions of a sensor may vary. In certain embodiments, the overall length of sensor strip 10, 10′ may be no less than about 20 mm and no greater than about 50 mm. For example, the length may be between about 30 and 45 mm; e.g., about 30 to 40 mm. It is understood, however that shorter and longer sensor strips 10, 10′ could be made. In certain embodiments, the overall width of sensor strip 10, 10′ may be no less than about 3 mm and no greater than about 15 mm. For example, the width may be between about 4 and 10 mm, about 5 to 8 mm, or about 5 to 6 mm. In one particular example, sensor strip 10, 10′ has a length of about 32 mm and a width of about 6 mm. In another particular example, sensor strip 10, 10′ has a length of about 40 mm and a width of about 5 mm. In yet another particular example, sensor strip 10, 10′ has a length of about 34 mm and a width of about 5 mm.

### Substrates

As provided above, sensor strip 10, 10′ has first and second substrates 12, 14, non-conducting, inert substrates which form the overall shape and size of sensor strip 10, 10′. Substrates 12, 14 may be substantially rigid or substantially flexible. In certain embodiments, substrates 12, 14 are flexible or deformable. Examples of suitable materials for substrates 12, 14 include, but are not limited, to polyester, polyethylene, polycarbonate, polypropylene, nylon, and other “plastics” or polymers. In certain embodiments the substrate material is “Melinex” polyester. Other non-conducting materials may also be used.

### Spacer Layer

As indicated above, positioned between substrate 12 and substrate 14 can be spacer 15 to separate first substrate 12 from second substrate 14. Spacer 15 is an inert non-conducting substrate, typically at least as flexible and deformable (or as rigid) as substrates 12, 14. In certain embodiments, spacer 15 is an adhesive layer or double-sided adhesive tape or film. Any adhesive selected for spacer 15 should be selected to not diffuse or release material which may interfere with accurate analyte measurement.

In certain embodiments, the thickness of spacer 15 may be at least about 0.01 mm (10 μm) and no greater than about 1 mm or about 0.5 mm. For example, the thickness may be between about 0.02 mm (20 μm) and about 0.2 mm (200 μm). In one certain embodiment, the thickness is about 0.05 mm (50 μm), and about 0.1 mm (100 μm) in another embodiment.

### Sample Chamber

The sensor includes a sample chamber for receiving a volume of sample to be analyzed; in the embodiment illustrated, particularly in FIG. 1, sensor strip 10, 10′ includes sample chamber 20 having an inlet 21 for access to sample chamber 20. In the embodiments illustrated, sensor strips 10, 10′ are side-fill sensor strips, having inlet 21 present on a side edge of strips 10, 10′, or on both side edges. Tip-fill and top-fill sensors can also be configured in accordance with this invention.

Sample chamber 20 is configured so that when a sample is provided in chamber 20, the sample is in electrolytic contact with both the working electrode and the counter electrode, which allows electrical current to flow between the electrodes to effect the electrolysis (electrooxidation or electroreduction) of the analyte.

Sample chamber 20 is defined by substrate 12, substrate 14 and spacer 15; in many embodiments, sample chamber 20 exists between substrate 12 and substrate 14 where spacer 15 is not present. Typically, a portion of spacer 15 is removed to provide an area between substrates 12, 14 without spacer 15; this volume of removed spacer is sample chamber 20. For embodiments that include spacer 15 between substrates 12, 14, the thickness of sample chamber 20 is generally the thickness of spacer 15.

Sample chamber 20 has a volume sufficient to receive a sample of biological fluid therein. In some embodiments, such as when sensor strip 10, 10′ is a small volume sensor, sample chamber 20 has a volume that is preferably no more than about 1 μL, for example no more than about 0.5 μL, and also for example, no more than about 0.25 μL. A volume of no more than about 0.1 μL is also suitable for sample chamber 20, as are volumes of no more than about 0.05 μL and about 0.03 μL.

As provided above, the thickness of sample chamber 20 corresponds typically to the thickness of spacer 15. Particularly for facing electrode configurations, this thickness is small to promote rapid electrolysis of the analyte, as more of the sample will be in contact with the electrode surface for a given sample volume.

### Electrodes

As provided above, the sensor includes a working electrode and at least one counter electrode. The counter electrode may be a counter/reference electrode. If multiple counter electrodes are present, one of the counter electrodes will be a counter electrode and one or more may be reference electrodes. Referring to FIGS. 2A and 2B and FIGS. 3A, 3B and 4, two examples of suitable electrode configurations are illustrated.

### Working Electrode

At least one working electrode is positioned on one of first substrate 12 and second substrate 14. In all of FIGS. 2A though 4, working electrode 22 is illustrated on substrate 12. Working electrode 22 extends from the sample chamber 20 to the other end of the sensor 10 as an electrode extension called a “trace”. The trace provides a contact pad 23 for providing electrical connection to a meter or other device to allow for data and measurement collection, as will be described later. Contact pad 23 can be positioned on a tab 26 that extends from the substrate on which working electrode 22 is positioned, such as substrate 12. In one embodiment, a tab has more than one contact pad positioned thereon. In a second embodiment, a single contact pad is used to provide a connection to one or more electrodes; that is, multiple electrodes are coupled together and are connected via one contact pad.

Working electrode 22 can be a layer of conductive material such as gold, carbon, platinum, ruthenium dioxide, palladium, or other non-corroding, conducting material. Working electrode 22 can be a combination of two or more conductive materials. An example of a suitable conductive epoxy is ECCOCOAT CT5079-3 Carbon-Filled Conductive Epoxy Coating (available from W.R. Grace Company, Woburn, Mass.). The material of working electrode 22 typically has relatively low electrical resistance and is typically electrochemically inert over the potential range of the sensor during operation.

Working electrode 22 may be applied on substrate 12 by any of various methods, including by being deposited, such as by vapor deposition or vacuum deposition or otherwise sputtered, printed on a flat surface or in an embossed or otherwise recessed surface, transferred from a separate carrier or liner, etched, or molded. Suitable methods of printing include screen-printing, piezoelectric printing, ink jet printing, laser printing, photolithography, and painting.

As provided above, at least a portion of working electrode 22 is provided in sample chamber 20 for the analysis of analyte, in conjunction with the counter electrode.

### Counter Electrode

The sensor includes at least one counter electrode positioned within the sample chamber. In FIGS. 2A and 2B, counter electrode 24 is illustrated on substrate 14. In FIGS. 3A, 3B and 4, a counter electrode 24 is present on substrate 12. Counter electrode 24 extends from the sample chamber 20 to the other end of the sensor 10 as an electrode extension called a “trace”. The trace provides a contact pad 25 for providing electrical connection to a meter or other device to allow for data and measurement collection, as will be described later. Contact pad 25 can be positioned on a tab 27 that extends from the substrate on which counter electrode 24 is positioned, such as substrate 12 or 14. In one embodiment, a tab has more than one contact pad positioned thereon. In a second embodiment, a single contact pad is used to provide a connection to one or more electrodes; that is, multiple electrodes are coupled together and are connected via one contact pad.

Counter electrode 24 may be constructed in a manner similar to working electrode 22. Suitable materials for the counter/reference or reference electrode include Ag/AgCl or Ag/AgBr on a non-conducting base material or silver chloride on a silver metal base. The same materials and methods may be used for counter electrode 24 as are available for working electrode 22, although different materials and methods may also be used. Counter electrode 24 can include a mix of multiple conducting materials, such as Ag/AgCl and carbon.

### Electrode Configurations

Working electrode 22 and counter electrode 24 may be disposed opposite to and facing each other to form facing electrodes. See for example, FIG. 2A, which has working electrode 22 on substrate 12 and counter electrode 24 on substrate 14, forming facing electrodes. In this configuration, the sample chamber is typically present between the two electrodes 22, 24. For this facing electrode configuration, electrodes 22, 24 may be separated by a distance of no more than about 0.2 mm (e.g., at least one portion of the working electrode is separated from one portion of the counter electrode by no more than about 200 μm), e.g., no more than about 100 μm, e.g., no more than about 50 μm.

Working electrode 22 and counter electrode 24 can alternately be disposed generally planar to one another, such as on the same substrate, to foam co-planar or planar electrodes. Referring to FIGS. 3A and 4, both working electrode 22 and counter electrode 24 occupy a portion of the surface of substrate 12, thus forming co-planar electrodes.

### Sensing Chemistry

In addition to working electrode 22, sensing chemistry material(s) are preferably provided in sample chamber 20 for the analysis of the analyte. Sensing chemistry material facilitates the transfer of electrons between working electrode 22 and the analyte in the sample. Any sensing chemistry may be used in sensor strip 10, 10′. Examples of sensing chemistry are described in, for example, U.S. Pat. Nos. 6,175,752, 6,461,496, 6,591,125, and 6,616,819, the disclosure of each of which are herein incorporated by reference for all purposes.

### Electron Transfer Agent

The sensing chemistry may include an electron transfer agent that facilitates the transfer of electrons to or from the analyte. The electron transfer agent may be present on working electrode 22 as a layer. One example of a suitable electron transfer agent is an enzyme which catalyzes a reaction of the analyte. For example, a glucose oxidase or glucose dehydrogenase, such as pyrroloquinoline quinone glucose dehydrogenase (PQQ), is used when the analyte is glucose. Other enzymes can be used for other analytes.

The electron transfer agent facilitates a current between working electrode 22 and the analyte and enables the electrochemical analysis of molecules. The agent facilitates the transfer electrons between the electrode and the analyte.

### Sorbent Material

Sample chamber 20 can be empty before the sample is placed in the chamber, or, in some embodiments, the sample chamber can include a sorbent material to sorb and hold a fluid sample during the measurement process. The sorbent material facilitates the uptake of small volume samples by a wicking action which can complement or, e.g., replace any capillary action of the sample chamber. Suitable sorbent materials include polyester, nylon, cellulose, and cellulose derivatives such as nitrocellulose. In addition to or alternatively, a portion or the entirety of the wall of the sample chamber may be coated by a surfactant, which is intended to lower the surface tension of the fluid sample and improve fluid flow within the sample chamber.

Methods other than the wicking action of a sorbent can be used to transport the sample into the sample chamber. Examples of such methods for transport include the application of pressure on a sample to push it into the sample chamber, the creation of a vacuum by a pump or other vacuum-producing method in the sample chamber to pull the sample into the chamber, capillary action due to interfacial tension of the sample with the walls of a thin sample chamber, as well as the wicking action of a sorbent material.

### Fill Indicator Electrode

In some instances, it is desirable to be able to determine when the sample chamber is filled. Sensor strip 10, 10′ can be indicated as filled, or substantially filled, by observing a signal between an indicator electrode and one or both of working electrode 22 or counter electrode 24 as sample chamber 20 fills with fluid. When fluid reaches the indicator electrode, the signal from that electrode will change. Suitable signals for observing include, for example, voltage, current, resistance, impedance, or capacitance between the indicator electrode and, for example, working electrode 22. Alternatively, the sensor can be observed after filling to determine if a value of the signal (e.g., voltage, current, resistance, impedance, or capacitance) has been reached indicating that the sample chamber is filled.

Typically, the indicator electrode is further downstream from a sample inlet, such as inlet 21, than working electrode 22 and counter electrode 24.

For side-fill sensors, an indicator electrode can be present on each side of the counter electrode. This permits the user to fill the sample chamber from either the left or right side with an indicator electrode disposed further upstream. This three-electrode configuration is not necessary. Side-fill sensors can also have a single indicator electrode and may include some indication as to which side should be placed in contact with the sample fluid.

The indicator electrode can also be used to improve the precision of the analyte measurements. The indicator electrode may operate as a working electrode or as a counter electrode or counter/reference electrode. Measurements from the indicator electrode/working electrode can be combined (for example, added or averaged) with those from the first counter/reference electrode/working electrode to obtain more accurate measurements.

The sensor or equipment that the sensor connected is with (e.g., a meter) can include a sign (e.g., a visual sign or auditory signal) that is activated in response to the indicator electrode to alert the user that the sample chamber has been filled. The sensor or equipment can be configured to initiate a reading when the indicator electrode indicates that the sample chamber has been filled with or without alerting the user. The reading can be initiated, for example, by applying a potential between the working electrode and the counter electrode and beginning to monitor the signals generated at the working electrode.

### Insertion Monitor

In accordance with various embodiments, the sensor includes an indicator to notify when proper insertion of sensor strip 10, 10′ into receiving equipment, such as a meter, has occurred. As seen in FIGS. 1, 2A, 2B, 3A and 3B, sensor strips 10, 10′ include insertion monitor 30 on an exterior surface of one of substrates 12, 14.

Insertion monitor 30 is used to encode information regarding sensor strip 10, 10′. The encoded information can be, for example, calibration information for that manufacturing lot or for that specific strip. Such calibration information or code may relate to, e.g., the sensitivity of the strip or to the y-intercept and/or slope of its calibration curve. The calibration code is used by the meter or other equipment to which sensor strip 10, 10′ is connected to provide an accurate analyte reading. For example, based on the calibration code, the meter uses one of several programs stored within the meter.

In some embodiments, a value indicative of the calibration code is manually entered into the meter or other equipment, for example, by the user. In other embodiments, the calibration code is directly read by the meter or other equipment, thus not requiring input or other interaction by the user.

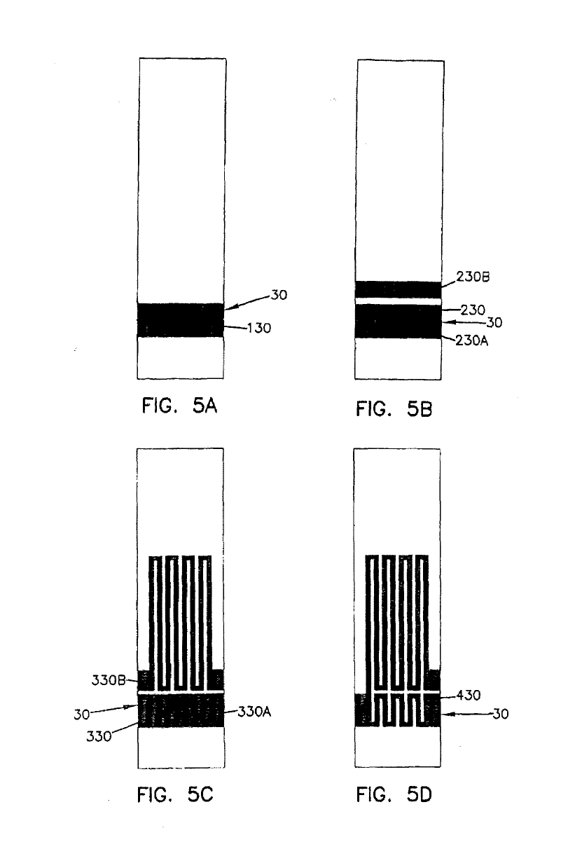

In one embodiment, illustrated, for example in FIG. 5A, insertion monitor 30 is a stripe 130 extending across an exterior surface of sensor 10, 10′, for example, from side edge to side edge or substantially so, with one contact pad for connection to a meter. It is understood that in alternate embodiments stripe 130 need not extend to both side edges. In another embodiment, the insertion monitor comprises two or more contact pads for connection to a meter. The two or more contact pads are electrically connected to each other by a material, such as a conductive ink.

The calibration code can be designed into insertion monitor 30, for example, either by the resistance or other electrical characteristic of insertion monitor 30, by the placement or position of insertion monitor 30, or by the shape or configuration of insertion monitor 30.

Insertion monitor 30 may alternately or additionally carry other information regarding the sensor strip 10, 10′. This other information that could be encoded into insertion monitor 30 include the test time needed for accurate analyte concentration analysis, expiration date of the sensor strip 10, 10′, various correction factors, such as for environmental temperature and/or pressure, selection of the analyte to be analyzed (e.g., glucose, ketone, lactate), and the like.

The resistance of insertion monitor 30, such as that of single stripe 130 or area or of a conductive path between the two or more contact pads, is related to the encoded information. As an example of discrete calibration values, resistance values in a given range can correspond to one calibration setting, and resistance values in a different range can correspond to a different calibration setting. Thus, when a meter or other equipment receives a sensor strip, indicator monitor 30 will notify the meter or equipment which assay calculation to use.

In addition to varying the resistance of indicator monitor 30 by varying the conductive or semi-conductive material used, the resistance of indicator monitor 30 can be varied by cutting or scoring some or all of the conductive pathways so that they do not carry charge. The resistance can additionally or alternately be controlled by the width or length of the conductive path. An example of a material suitable for indicator monitor 30 is a combination of carbon and silver; the resistance of this mixture will vary, based on the ratio of the two materials.

The placement or position of insertion monitor 30 can additionally or alternately be related to the encoded calibration information. For example, the calibration code can be directly related to the location of indicator monitor 30. For example, the position of indicator monitor 30 can be varied so that is makes electrical contact with different contact structures. (Contact structures are described below in “Sensor Connection to Electrical Device”). Depending on the contact structures engaged, the meter will recognize the calibration code and thus know what parameter to use to calculate an accurate analyte level.

The shape and/or configuration of insertion monitor 30 can additionally or alternatively be related to the encoded calibration code. For example, the calibration code can be directed related to which and/or the number of contact structures that make electrical contact with indicator monitor 30. For example, a pattern of discrete and unconnected indicator monitors can be present on the sensor; the calibration code will be directly related to the arrangement of those monitors. The pattern could be parallel lines, orderly arranged dots or squares, or the like.

While it is preferred to provide this encoded information on the insertion monitor, it should be recognized that the insertion monitor function and the encoding of information can also be implemented separately using separate conductive traces on the strip.

Conductive insertion monitor 30 is positioned on the non-conductive base substrate and has a contact pad for electrical contact with a connector. Insertion monitor 30 is configured and arranged to close an electrical circuit when sensor 10, 10′ is properly inserted into the connector.

Insertion monitor 30 may have any suitable configuration, including but not limited to, a stripe extending across sensor strip 10, 10′ from a side edge to a side edge, such as stripe 130, a stripe extending across the sensor strip, although not the entire width, and an array of unconnected dots, strips, or other areas. Other suitable configurations for insertion monitor 30 are illustrated in FIGS. 5B, 5C and 5D. FIG. 5B illustrates insertion monitor 30 as bi-regional monitor 230, having a first stripe 230A and a second stripe 230B, both of which extend from side edge to side edge, although it is understood that one or both of strips 230A, 230B may not extend completely to a side edge. FIGS. 5C and 5D illustrate insertion monitors that have a long, tortuous path, which extends longitudinally toward an end of the sensor, rather than extending merely side-to-side. Insertion monitor 330 of FIG. 5C has a stripe 330A and an elongate stripe 330B. Insertion monitor 430 of FIG. 5D has a single conductive strip 430, which provides an elongate path.

### Sensor Connection to Electrical Device

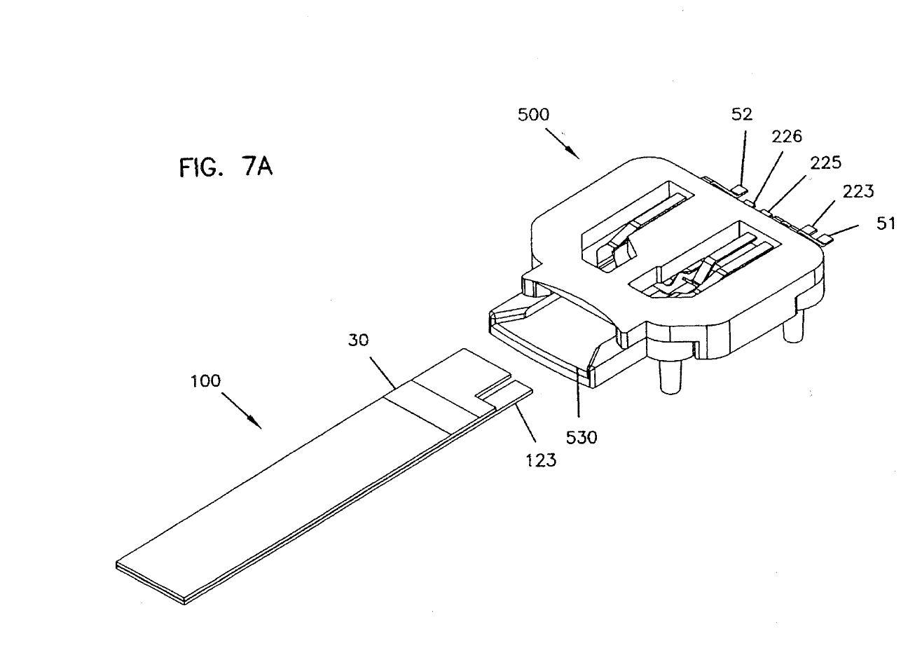

Referring to FIGS. 7A-7B, a sensor strip 100 is illustrated readied for insertion into a connector 500. Sensor strip 100 is similar to sensor strips 10, 10′. Sensor strip 100 includes insertion monitor 30 on an outer surface of one of the substrates forming strip 100. Sensor strip 100 includes, although not illustrated, one working electrode and three counter electrodes. The working electrode includes a contact pad positioned on tab 123 (see FIG. 7A).

Sensor strip 100 is configured to couple to a meter or other electrical device by electrical connector 500 which is configured to couple with and contact the end of sensor 100 at contact pads. The sensor meter typically includes a potentiostat or other component to provide a potential and/or current for the electrodes of the sensor. The sensor reader also typically includes a processor (e.g., a microprocessor or hardware) for determining analyte concentration from the sensor signals. The sensor meter also includes a display or a port for coupling a display to the sensor. The display displays the sensor signals and/or results determined from the sensor signals including, for example, analyte concentration, rate of change of analyte concentration, and/or the exceeding of a threshold analyte concentration (indicating, for example, hypo- or hyperglycemia).

One example of a suitable connector is shown in FIGS. 7A and 7B. Connector 500 (which is used to connect a sensor to a meter or other electrical device) is generally a two part structure, having top portion 510 and bottom portion 520 (see FIG. 7B). Positioned between and secured by top portion 510 and bottom portion 520 are various contact leads that provide electrical connection between sensor 100 and a meter. Bottom portion includes leads which have proximal ends to physically contact pads, and to connect to any attached meter. The end of sensor 100 having the contact pads can be slid into or mated with connector 500 by placing sensor 100 into slide area 530, which provides a support for and retains sensor 100. It is typically important that the contact structures of the connector 500 make electrical contact with the correct pads of the sensor so that the working electrode and counter electrode(s) are correctly coupled to the meter.

Connector 500 includes leads or contact structures 51, 52 for connection to insertion monitor 30. Insertion monitor 30 is configured and arranged to close an electrical circuit between contact structures 51 and 52 when the sensor is properly inserted into the connector. Proper insertion into connector 500 means that the sensor strip 100 is inserted right side up, that the correct end of strip 100 is inserted into connector 500, and that sensor strip 100 is inserted far enough into connector 500 that reliable electrical connections are made. Preferably, no closed circuit is made unless all electrode pads have properly contacted the contact structures of connector 500. The insertion monitor may have shapes other than a stripe across the width of the sensor; for example, other designs include an individual dot, a grid pattern, or may include stylistic features, such as words or letters.

Because this insertion monitor 30 is not at the end with the contact regions for the electrodes, the insertion monitor 30 does not require additional width space on the sensor.

In an optional embodiment to ensure proper insertion of a sensor into a meter, the meter may include a raised area or bump that prevents or hinders the insertion of the sensor in an improper direction. Objects other than a raised area can also be used to guide the user in correct introduction of the sensor into the meter.

### Illustrative Method for Manufacturing Sensors

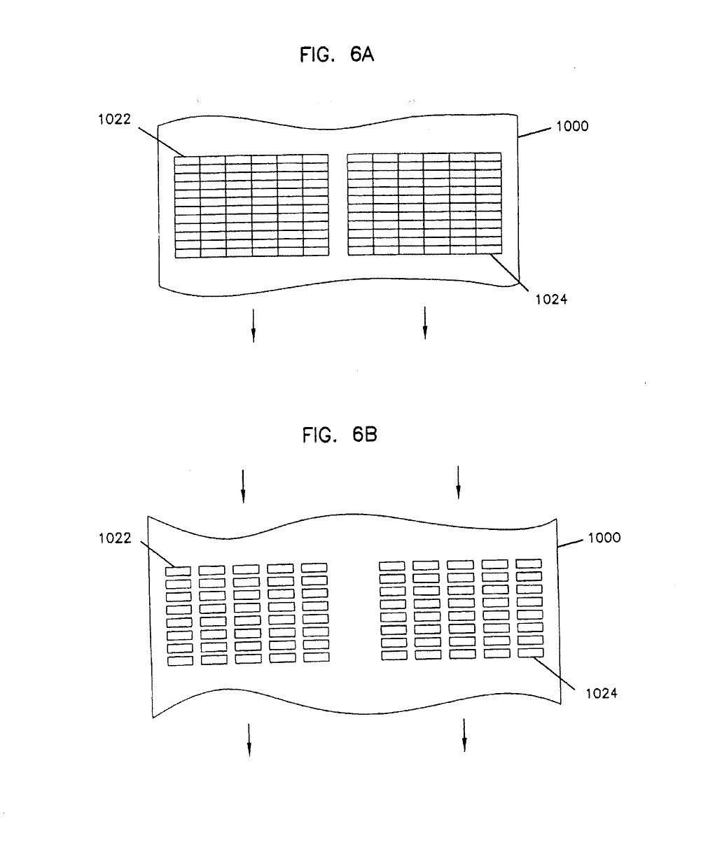

Referring now to FIGS. 6A and 6B, one example of a method for making sensors having two substrates with electrodes thereon is described with respect to the sensor arrangement displayed in FIG. 2A, although this method can be used to make a variety of other sensor arrangements, including those described before. When the three layers of FIG. 2A are assembled, a sensor similar to sensor 10 is formed.

In FIGS. 6A and 6B, a substrate 1000, such as a plastic substrate, is moving in the direction indicated by the arrow. Substrate 1000 can be an individual sheet or a continuous roll on a web. Multiple sensors can be formed on substrate 1000 as sections 1022 that have working electrodes 22 (FIG. 2A) thereon and sections 1024 that have counter electrodes 24 (FIG. 2A) thereon and other electrodes, such as reference electrodes and/or fill indicator electrodes. These working, counter and optional electrodes are electrically connected to their corresponding traces and contact pads. Typically, working electrode sections 1022 are produced on one half of substrate 1000 and counter electrode sections 1024 are produce on the other half of substrate 1000. In some embodiments, substrate 1000 can be scored and folded to bring the sections 1022, 1024 together to form the sensor. In some embodiments, as illustrated in FIG. 6A, the individual working electrode sections 1022 can be formed next to or adjacent each other on substrate 1000, to reduce waste material. Similarly, individual counter electrode sections 1024 can be formed next to or adjacent each other. In other embodiments, the individual working electrode sections 1022 (and, similarly, the counter electrode sections 1024) can be spaced apart, as illustrated in FIG. 6B. The remainder of the process is described for the manufacture of multiple sensors, but can be readily modified to form individual sensors.

Carbon or other electrode material (e.g., metal, such as gold or platinum) is formed on substrate 1000 to provide a working electrode 22 for each sensor. The carbon or other electrode material can be deposited by a variety of methods including printing a carbon or metal ink, vapor deposition, and other methods. The printing may be done by screen printing, gravure roll printing, transfer printing, and other known printing methods. The respective trace and contact pad 23 could be applied together with working electrode 22, but may be applied in a subsequent step.

Similar to the working electrode 22, counter electrode 24 is formed on substrate 1000. The counter electrode(s) are formed by providing carbon or other conductive electrode material onto substrate 1000. In one embodiment, the material used for the counter electrode(s) is a Ag/AgCl ink. The material of the counter electrode(s) may be deposited by a variety of methods including printing or vapor deposition. The printing may be done by screen printing, gravure roll printing, transfer printing, and other known printing methods. The respective trace and contact pad 25 could be applied together with counter electrodes 24, but may be applied in a subsequent step.

Preferably, multiple sensors 10 are manufactured simultaneously; that is, the working electrodes, including their traces and contact pads, for a plurality of sensors are produced (e.g., printed) on a polymer sheet or web, and simultaneously or subsequently, the counter electrodes, and their traces and contact pads, for a plurality of sensors are produced (e.g., printed). The working electrode(s) and counter electrode(s) can be formed on separate substrates that are later positioned opposite one another so that the electrodes face each other. Alternately, to simplify registration of the substrates, the working electrodes can be formed on a first half of a substrate sheet of web and the counter electrodes are formed on a second half of the substrate sheet or web so that the sheet or web can be folded to superimpose the working and counter electrodes in a facing arrangement.

To provide sample chamber 20, spacer 15 is formed over at least one of the substrate/working electrode and substrate/counter electrode(s). Spacer 15 can be an adhesive spacer, such as a single layer of adhesive or a double-sided adhesive tape (e.g., a polymer carrier film with adhesive disposed on opposing surfaces). Suitable spacer materials include adhesives such as urethanes, acrylates, acrylics, latexes, rubbers and the like.

A channel, which will result in the sample chamber, is provided in spacer 15, either by cutting out a portion of the adhesive spacer or placing two adhesive pieces in close proximity but having a gap therebetween. The adhesive can be printed or otherwise disposed on the substrate according to a pattern which defines the channel region. The adhesive spacer can be optionally provided with one or more release liners prior to its incorporation into the sensor. The adhesive can be cut (e.g., die-cut or slit) to remove the portion of the adhesive corresponding to the channel prior to disposing the spacer on the substrate.

The sides of the sensor can be straight to allow the sensor to be cut out from the remainder of the substrate and/or from other sensors by slitting the substrate in parallel directions using, for example, a gang arbor blade system. The edges of the sensor can define edges of the sample chamber. By accurately controlling the distance between cuts, variability in sample chamber volume can often be reduced. In some instances, these cuts are parallel to each other, as parallel cuts are typically the easiest to reproduce.

### Application of the Sensor

A common use for the analyte sensor, such as sensor strip 10, 10′, 100 is for the determination of analyte concentration in a biological fluid, such as glucose concentration in blood, interstitial fluid, and the like, in a patient or other user. Sensor strips 10, 10′, 100 may be available at pharmacies, hospitals, clinics, from doctors, and other sources of medical devices. Multiple sensor strips 10, 10′, 100 may be packaged together and sold as a single unit; e.g., a package of 25, 50, or 100 strips.

Sensor strips 10, 10′, 100 can be used for an electrochemical assay, or, for a photometric test. Sensor strips 10, 10′, 100 are generally configured for use with an electrical meter, which may be connectable to various electronics. A meter may be available at generally the same locations as sensor strips 10, 10′, 100 and sometimes may be packaged together with sensor strips 10, 10′, 100, e.g., as a kit.

Examples of suitable electronics connectable to the meter include a data processing terminal, such as a personal computer (PC), a portable computer such as a laptop or a handheld device (e.g., personal digital assistants (PDAs)), and the like. The electronics are configured for data communication with the receiver via a wired or a wireless connection. Additionally, the electronics may further be connected to a data network (not shown) for storing, retrieving and updating data corresponding to the detected glucose level of the user.

The various devices connected to the meter may wirelessly communicate with a server device, e.g., using a common standard such as 802.11 or Bluetooth RF protocol, or an IrDA infrared protocol. The server device could be another portable device, such as a Personal Digital Assistant (PDA) or notebook computer, or a larger device such as a desktop computer, appliance, etc. In some embodiments, the server device does have a display, such as a liquid crystal display (LCD), as well as an input device, such as buttons, a keyboard, mouse or touch-screen. With such an arrangement, the user can control the meter indirectly by interacting with the user interface(s) of the server device, which in turn interacts with the meter across a wireless link.

The server device can also communicate with another device, such as for sending glucose data from the meter and/or the service device to a data storage or computer. For example, the service device could send and/or receive instructions (e.g., an insulin pump protocol) from a health care provider computer. Examples of such communications include a PDA synching data with a personal computer (PC), a mobile phone communicating over a cellular network with a computer at the other end, or a household appliance communicating with a computer system at a physician's office.

A lancing device or other mechanism to obtain a sample of biological fluid, e.g., blood, from the patient or user may also be available at generally the same locations as sensor strips 10 and the meter, and sometimes may be packaged together with sensor strips 10 and/or meter, e.g., as a kit.

### Integrated Sample Acquisition and Analyte Measurement Device

An analyte measurement device constructed according to the principles of the present invention typically includes a sensor strip 10, as described hereinabove, combined with a sample acquisition apparatus to provide an integrated sampling and measurement device. The sample acquisition apparatus typically includes, for example, a skin piercing member, such as a lancet, that can be injected into a patient's skin to cause blood flow. The integrated sample acquisition and analyte measurement device can comprise a lancing instrument that holds a lancet and sensor strip 10. The lancing instrument might require active cocking. By requiring the user to cock the device prior to use, the risk of inadvertently triggering the lancet is minimized. The lancing instrument could also permit the user to adjust the depth of penetration of the lancet into the skin. Such devices are commercially available from companies such as Boehringer Mannheim and Palco. This feature allows users to adjust the lancing device for differences in skin thickness, skin durability, and pain sensitivity across different sites on the body and across different users.

In one embodiment, the lancing instrument and the meter are integrated into a single device. To operate the device the user need only insert a disposable cartridge containing a sensor strip and lancing device into the integrated device, cock the lancing instrument, press it against the skin to activate it, and read the result of the measurement. Such an integrated lancing instrument and test reader simplifies the testing procedure for the user and minimizes the handling of body fluids.

In some embodiments, sensor strips 10 may be integrated with both a meter and a lancing device. Having multiple elements together in one device reduces the number of devices needed to obtain an analyte level and facilitates the sampling process.

For example, embodiments may include a housing that includes one or more of the subject strips, a skin piercing element and a processor for determining the concentration of an analyte in a sample applied to the strip. A plurality of strips 10 may be retained in a cassette in the housing interior and, upon actuation by a user, a single strip 10 may be dispensed from the cassette so that at least a portion extends out of the housing for use.

### Operation of the Sensor Strip

In use, a sample of biological fluid is provided into the sample chamber of the sensor, where the level of analyte is determined. The analysis may be based on providing an electrochemical assay or a photometric assay. In many embodiments, it is the level of glucose in blood that is determined. Also in many embodiments, the source of the biological fluid is a drop of blood drawn from a patient, e.g., after piercing the patient's skin with a lancing device, which could be present in an integrated device, together with the sensor strip.

The analyte in the sample is, e.g., electrooxidized or electroreduced, at working electrode 22, and the level of current obtained at counter electrode 24 is correlated as analyte concentration.

Sensor strip 10, 10′, 100 may be operated with or without applying a potential to electrodes 22, 24. In one embodiment, the electrochemical reaction occurs spontaneously and a potential need not be applied between working electrode 22 and counter electrode 24. In another embodiment, a potential is applied between working electrode 22 and counter electrode 24.

In some currently available systems, a value indicative of the calibration code of a sensor is manually entered into the meter or other equipment, for example, by the user. Based on the calibration code, the meter uses one of several programs or parameters stored within the meter. In other currently available systems, the sensor calibration code is directly read by the meter or other equipment, thus not requiring input or other interaction by the user. These sensors, however, still have a calibration code associated with them, which includes slope and y-intercept values. The slope and y-intercept values are used to determine the analyte concentration based on the measured signal. The calibration code, whether inputted manually or automatically, is needed to standardize the analysis results received from non-standardized sensors. In other words, different sensors vary, e.g., from lot to lot, a sufficient amount that, if no compensation were made, the results would differ from sensor to sensor and the results could be clinically inaccurate.

The sensors of this disclosure are calibration-adjusted to a pre-determined calibration (slope and y-intercept), during the manufacturing process, to avoid the need for the user to input or otherwise set a calibration code for the sensor or perform other calibration procedure(s) before using the sensor. The sensors of this disclosure are also calibration-adjusted to avoid the need for the meter to read a calibration code.

It has been determined that the measured signal (e.g., charge due to electrooxidation or electroreduction) from the analyte in a sample is proportional to a physical element of the sensor. For example, when coulometry is used to obtain a signal proportionate to the analyte concentration, the signal obtained is proportional to the volume of sample being assayed. For amperometry or other kinematical electrolysis, the signal is proportion to the area of the electrode(s), e.g., the at least one working electrode, in the sample chamber. By physically altering the sensor's sample chamber volume or electrode area within the sample chamber during the manufacturing process, e.g., after the assembly of multiple layers, the slope and y-intercept of the sensor lot can be controlled, e.g., shifted, to provide a sensor with a pre-determined calibration. In some embodiments, the relationship between the sample chamber volume and the measured signal is linear. Additionally or alternatively, in some embodiments the relationship between the electrode area and the measured signal is linear.

This disclosure also provides methods for making sensors that avoid the need for the user to input or otherwise set a calibration code for the sensor, or perform other calibration procedure(s) before using the sensor.

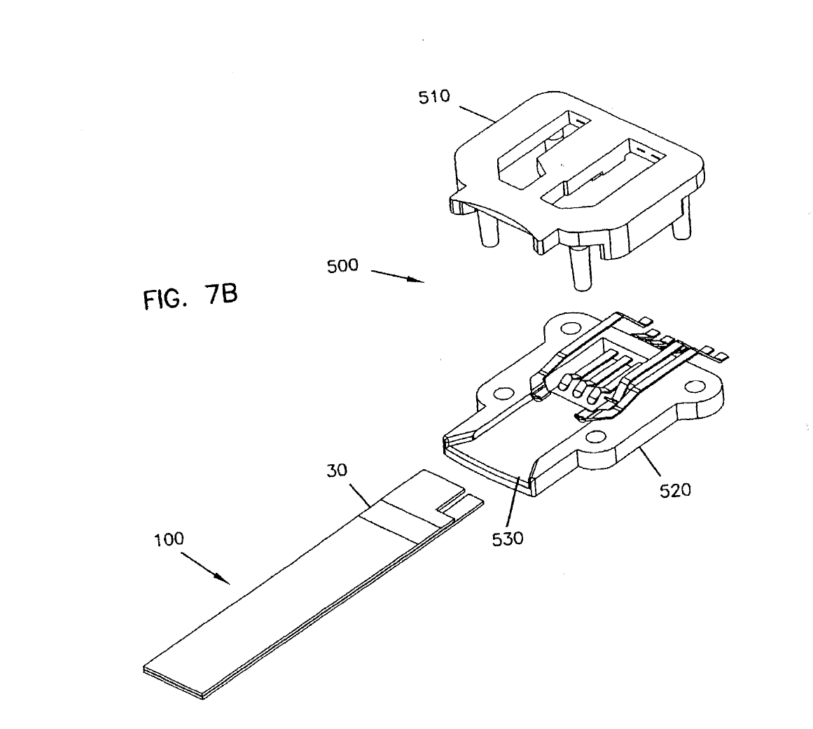

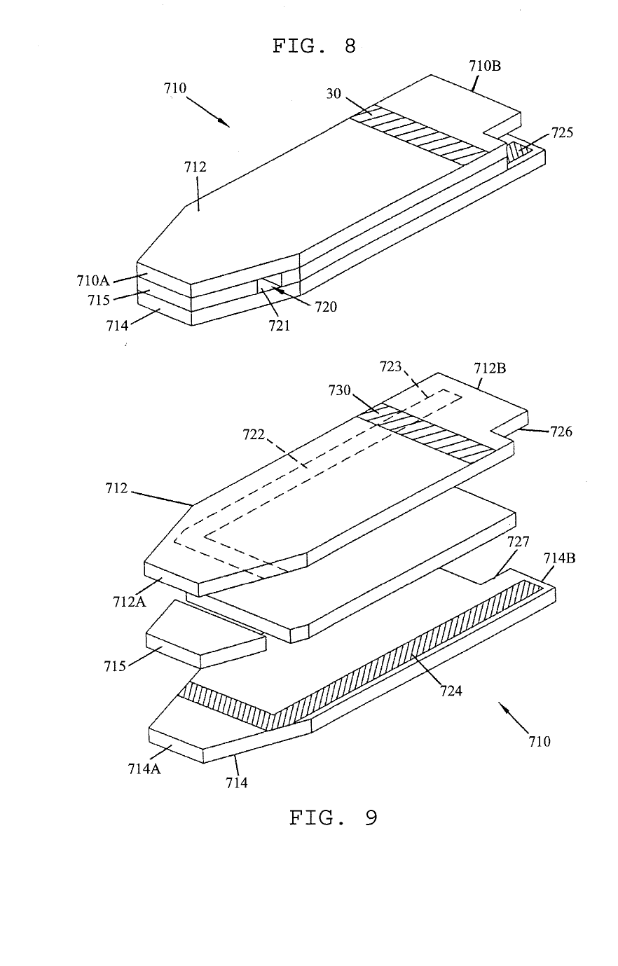

Referring to the Drawings in general and FIGS. 8 and 9 in particular, a first embodiment of a sensor 710 is schematically illustrated, herein shown in the shape of a strip. It is to be understood that the sensor may be any suitable shape. Sensor strip 710 has a first substrate 712, a second substrate 714, and a spacer 715 positioned therebetween.

Sensor strip 710 includes at least one working electrode 722 and at least one counter electrode 724. Sensor strip 710 also includes an optional insertion monitor 730. Sensor strip 710 has a first, distal end 710A and an opposite, proximal end 710B. At distal end 710A, sample to be analyzed is applied to sensor 710. Distal end 710A could be referred as ‘the fill end’, ‘sample receiving end’, or similar. Proximal end 710B of sensor 710 is configured for operable, and usually releasable, connecting to a device such as a meter.

Sensor strip 710 is a layered construction, in certain embodiments having a generally rectangular shape, i.e., its length is longer than its width, although other shapes 710 are possible as well, as noted above. The length of sensor strip 710 is from end 710A to end 710B.

The dimensions of a sensor may vary. In certain embodiments, the overall length of sensor strip 710 may be no less than about 10 mm and no greater than about 50 mm. For example, the length may be between about 30 and 45 mm; e.g., about 30 to 40 mm. It is understood, however that shorter and longer sensor strips 710 could be made. In certain embodiments, the overall width of sensor strip 710 may be no less than about 3 mm and no greater than about 15 mm. For example, the width may be between about 4 and 10 mm, about 5 to 8 mm, or about 5 to 6 mm. In one particular example, sensor strip 710 has a length of about 32 mm and a width of about 6 mm. In another particular example, sensor strip 710 has a length of about 40 mm and a width of about 5 mm. In yet another particular example, sensor strip 710 has a length of about 34 mm and a width of about 5 mm.

### Substrates and Spacer

As provided above, sensor strip 710 has first and second substrates 712, 714, non-conducting, inert substrates which form the overall shape and size of sensor strip 710. Substrates 712, 714 may be substantially rigid or substantially flexible. In certain embodiments, substrates 712, 714 are flexible or deformable. Examples of suitable materials for substrates 712, 714 include, but are not limited, to polyester, polyethylene, polycarbonate, polypropylene, nylon, and other “plastics” or polymers. In certain embodiments the substrate material is “Melinex” polyester. Other non-conducting materials may also be used.

Substrate 712 includes first or distal end 712A and second or proximal end 712B, and substrate 714 includes first or distal end 714A and second or proximal end 714B.

As indicated above, positioned between substrate 712 and substrate 714 may be spacer 715 to separate first substrate 712 from second substrate 714. In some embodiments, spacer 715 extends from end 710A to end 710B of sensor strip 710, or extends short of one or both ends. Spacer 715 is an inert non-conducting substrate, typically at least as flexible and deformable (or as rigid) as substrates 712, 714. In certain embodiments, spacer 715 is an adhesive layer or double-sided adhesive tape or film that is continuous and contiguous. Any adhesive selected for spacer 715 should be selected to not diffuse or release material which may interfere with accurate analyte measurement.

In certain embodiments, the thickness of spacer 715 may be constant throughout, and may be at least about 0.01 mm (10 μm) and no greater than about 1 mm or about 0.5 mm. For example, the thickness may be between about 0.02 mm (20 μm) and about 0.2 mm (200 μm). In one certain embodiment, the thickness is about 0.05 mm (50 μm), and about 0.1 mm (100 μm) in another embodiment.

### Sample Chamber

The sensor includes a sample chamber for receiving a volume of sample to be analyzed; in the embodiment illustrated, particularly in FIG. 8, sensor strip 710 includes sample chamber 720 having an inlet 721 for access to sample chamber 720. In the embodiment illustrated, sensor strip 710 is a side-fill sensor strip, having inlet 721 present on a side edge of strip 710. Tip-fill sensors, having an inlet at, for example, end 710A, are also within the scope of this disclosure, as well as corner and top filling sensors. Sample chamber 720 is configured so that when a sample is provided in chamber 720, the sample is in electrolytic contact with both a working electrode and a counter electrode, which allows electrical current to flow between the electrodes to effect the electrolysis (electrooxidation or electroreduction) of the analyte.

Sample chamber 720 is defined by substrate 712, substrate 714 and spacer 715; in many embodiments, sample chamber 720 exists between substrate 712 and substrate 714 where spacer 715 is not present. Typically, a portion of spacer 715 is removed to provide a volume between substrates 712, 714 without spacer 715; this volume of removed spacer is sample chamber 720. For embodiments that include spacer 715 between substrates 712, 714, the thickness of sample chamber 720 is generally the thickness of spacer 715.

Sample chamber 720 has a volume sufficient to receive a sample of biological fluid therein. In some embodiments, such as when sensor strip 710 is a small volume sensor, sample chamber 720 has a volume that is typically no more than about 1 μL, for example no more than about 0.5 μL, and also for example, no more than about 0.25 μL. A volume of no more than about 0.1 μL, is also suitable for sample chamber 720, as are volumes of no more than about 0.05 μL and about 0.03 μL.

As provided above, the thickness of sample chamber 720 corresponds typically to the thickness of spacer 715. Particularly for facing electrode configurations, as in the sensor illustrated in FIG. 9, this thickness is small to promote rapid electrolysis of the analyte, as more of the sample will be in contact with the electrode surface for a given sample volume.

### Electrodes

As provided above, the sensor includes a working electrode and at least one counter electrode. The counter electrode may be a counter/reference electrode. If multiple counter electrodes are present, one of the counter electrodes will be a counter electrode and one or more may be reference electrodes.

For sensor 710, at least one working electrode is positioned on one of first substrate 712 and second substrate 714 in the sample chamber. In FIG. 9, working electrode 722 is illustrated on substrate 712. Working electrode 722 extends from the sample chamber 720, proximate distal end 710A, to the other end of the sensor 710, end 710B, as an electrode extension called a “trace”. The trace provides a contact pad 723 for providing electrical connection to a meter or other device to allow for data and measurement collection, as will be described later. Contact pad 723 may be positioned on a tab 726 that extends from the substrate on which working electrode 722 is positioned, such as substrate 712. In some embodiments, a tab has more than one contact pad positioned thereon. In alternate embodiments, a single contact pad is used to provide a connection to one or more electrodes; that is, multiple electrodes are coupled together and are connected via one contact pad.

Working electrode 722 may be a layer of conductive material such as gold, carbon, platinum, ruthenium dioxide, palladium, or other non-corroding, conducting material. Working electrode 722 may be a combination of two or more conductive materials. An example of a suitable conductive epoxy is ECCOCOAT CT5079-3 Carbon-Filled Conductive Epoxy Coating (available from W.R. Grace Company, Woburn, Mass.). The material of working electrode 22 typically has relatively low electrical resistance and is typically electrochemically inert over the potential range of the sensor during operation.

Working electrode 722 may be applied on substrate 712 by any of various methods, including by being deposited, such as by vapor deposition or vacuum deposition or otherwise sputtered, printed on a flat surface or in an embossed or otherwise recessed surface, transferred from a separate carrier or liner, etched, or molded. Suitable methods of printing include screen-printing, piezoelectric printing, ink jet printing, laser printing, photolithography, and painting.

As provided above, at least a portion of working electrode 722 is provided in sample chamber 720 for the analysis of analyte, in conjunction with the counter electrode.

The sensor includes at least one counter electrode positioned within the sample chamber. In FIG. 9, counter electrode 724 is illustrated on substrate 714. In alternate embodiments, a counter electrode is present on a different surface or substrate, such as substrate 712. Counter electrode 724 extends from the sample chamber 720, proximate first end 710A, to the other end of the sensor 710, end 710B, as an electrode extension called a “trace”. The trace provides a contact pad 725 for providing electrical connection to a meter or other device to allow for data and measurement collection, as will be described later. Contact pad 725 may be positioned on a tab 727 that extends from the substrate on which counter electrode 724 is positioned, such as substrate 712 or 714. In some embodiments, a tab has more than one contact pad positioned thereon. In alternate embodiments, a single contact pad is used to provide a connection to one or more electrodes; that is, multiple electrodes are coupled together and are connected via one contact pad.

Counter electrode 724 may be constructed in a manner similar to working electrode 722. Suitable materials for the counter/reference or reference electrode include Ag/AgCl or Ag/AgBr on a non-conducting base material or silver chloride on a silver metal base. The same materials and methods may be used for counter electrode 724 as are available for working electrode 722, although different materials and methods may also be used. Counter electrode 724 may include a mix of multiple conducting materials, such as Ag/AgCl and carbon.

Working electrode 722 and counter electrode 724 may be disposed opposite to and facing each other to form facing electrodes. See for example, FIG. 9, which has working electrode 722 on substrate 712 and counter electrode 724 on substrate 714, forming facing electrodes. In this configuration, the sample chamber is typically present between the two electrodes 722, 724. Working electrode 722 and counter electrode 724 may alternately be positioned generally planar to one another, such as on the same substrate, to form co-planar or planar electrodes.

In some instances, it is desirable to be able to determine when the sample chamber of the sensor is sufficiently filled with sample. Sensor strip 710 may be indicated as filled, or substantially filled, by observing a signal between an optional indicator electrode and one or both of working electrode 722 or counter electrode 724 as sample chamber 720 fills with fluid. When fluid reaches the indicator electrode, the signal from that electrode will change. Suitable signals for observing include, for example, voltage, current, resistance, impedance, or capacitance between the indicator electrode and, for example, working electrode 722. Alternatively, the sensor may be observed after filling to determine if a value of the signal (e.g., voltage, current, resistance, impedance, or capacitance) has been reached indicating that the sample chamber is filled.

Typically, the indicator electrode is further downstream from a sample inlet, such as inlet 721, than working electrode 722 and/or counter electrode 724.

For side-fill sensors, such as sensor 710 of FIGS. 8 and 9, an indicator electrode may be present on each side of the counter electrode. This permits the user to fill the sample chamber from either the left or right side with an indicator electrode disposed further upstream. This three-electrode configuration is not necessary. Side-fill sensors may also have a single indicator electrode and may include some indication as to which side should be placed in contact with the sample fluid.

The indicator electrode may also be used to improve the precision of the analyte measurements. The indicator electrode may operate as a working electrode or as a counter electrode or counter/reference electrode. Measurements from the indicator electrode/working electrode may be combined (e.g., added or averaged) with those from the first counter/reference electrode/working electrode to obtain more accurate measurements.

The sensor or equipment that the sensor connected is with (e.g., a meter) may include a signal (e.g., a visual sign or auditory tone) that is activated in response to activation of the indicator electrode to alert the user that the desired zone has been filled. The sensor or equipment may be configured to initiate a reading when the indicator electrode indicates that the sample chamber has been filled with or without alerting the user. The reading may be initiated, for example, by applying a potential between the working electrode and the counter electrode and beginning to monitor the signals generated at the working electrode.

### Insertion Monitor

The sensor may include an indicator to notify when proper insertion of the sensor into receiving equipment, such as a meter, has occurred. As seen in FIGS. 8 and 9, sensor strip 710 includes insertion monitor 730 on an exterior surface of one of substrates 712, 714, in the illustrated sensor, sensor 710. Insertion monitor 730 is configured and arranged to close an electrical circuit when sensor 710 is properly inserted into the meter connector.

Insertion monitor 730 may be a stripe extending across an exterior surface of sensor 710, for example, from side edge to side edge, with one contact pad for connection to a meter. It is understood that in alternate embodiments of the insertion monitor, the stripe need not extend to both side edges. In other embodiments, the insertion monitor may be two or more contact pads for connection to a meter. The two or more contact pads could electrically connected to each other by a material, such as a conductive ink.

Insertion monitor 730 can be used to encode information regarding sensor strip 710. The encoded information may be, for example, the test time needed for accurate analyte concentration analysis, the expiration date of sensor strip 710, various correction factors, such as for environmental temperature and/or pressure, selection of the analyte to be analyzed (e.g., glucose, ketone, lactate), and the like. Additionally, insertion monitor 730 can be used to encode calibration information for the sensor, e.g., for the manufacturing lot or that specific sensor strip. However, in accordance with this disclosure, the sensor requires no calibration code; rather, the sensor is configured with a pre-determined calibration, based on the volume of the sample chamber.

Additional details regarding insertion monitors, and their use for encoding information, are described, for example, in U.S. Patent application publication no. 2006/0091006 A1, and at U.S. Pat. Nos. 6,503,381 and 6,773,671, hereby incorporated by reference. Additionally, U.S. Patent application publication no. 2006/0091006 A1 provides various details regarding connection of sensors with insertion monitors with meters and connectors.

### General Method for Manufacturing Sensors

Sensor strips 710 discussed above, are sandwiched or layered constructions having substrates 712, 714 spaced apart, such as by spacer 715. Such a construction may be made by laminating the various layers together, in any suitable manner. An alternate method for making sensor strips 710, and other sensors in accordance with the invention, is to mold the sensors.

Molding may include positioning at least two spaced apart electrically conductive electrodes (e.g., wires) in a mold, and molding a body of insulative material around the electrodes, with one end having therein means for receiving a fluid sample. More specifically, molding could include positioning at least two spaced apart electrically conductive electrodes (e.g., wires) in a mold, before or after molding, treating at least one of the electrodes with one or more chemicals to change the electrical properties of the treated electrode upon contact with a fluid sample, and molding a body of insulative material around the electrodes with one end having therein means for receiving a fluid sample. The body may be molded in multiple pieces, e.g., two pieces, with a body and end cap for attaching to one another after the molding is completed, or in a single piece.

A sensor may be made by positioning electrodes on one or more substrates, the substrates including a first substrate, optionally contacting at least a portion of at least one electrode with sensing material(s), and configuring the sensor by positioning a spacer between the two substrates to maintain the substrates in a fixed, layered orientation relative to each other.

### Calibration of Sensors

Whether the sensors are laminated, molded, or made by some other process, after or during forming the sensor, a portion of the sensor is physically modified (e.g., removed, re-shaped, reacted, etc.) to provide the sensor with a pre-determined slope and y-intercept. Typically, the physically modified portion of the sensor includes the sample chamber. In accordance with some embodiments of this disclosure, the sample chamber shape and/or size is altered to provide the sensor with the desired pre-determined slope and y-intercept. In many embodiments, the shape and/or size of the sample chamber is physically modified. Additionally or alternately, in accordance with some embodiments of this disclosure, the electrode area within the sample chamber is altered, sometimes without altering the sample chamber shape and/or size. In many embodiments, the electrode area is physically modified.

It is noted that in alternate embodiments, the sample chamber volume may remain the same although the area of the electrodes is modified. One exemplary method for modifying the electrode area, e.g., removing area, is by the use of non-invasive procedures, such as a single or multiple energy beams (e.g., lasers, UV light, electron beam, etc.) that pass through the inert substrates but physically alter the electrodes. In this process, areas of electrode(s) may be removed or otherwise rendered inactive.

To provide a plurality of sensors, such as sensor strips 710 with the same pre-determined calibration from a plurality of pre-sensors, each of the pre-sensors may be physically altered, as needed, to obtain the desired pre-determined physical characteristics and the desired sensor. It is understood that this discussion also applies to a batch or lot of sensors in addition to a single sensor. For example, a first pre-sensor may have a response that is too high compared to the desired level and a second pre-sensor may have a response that is within the desired level. In such a situation, a portion of the first pre-sensor may be removed to provide a sensor having a, sample chamber, or electrode area that is comparable to that of the second pre-sensor and is within the desired level.

In some situations, however, a pre-sensor, e.g., a third sensor strip, may have a response that is too low compared to the desired level. Because in most embodiments it would be difficult or impractical to increase the sample chamber, and/or electrode area of the pre-sensor after it has been assembled, in some manufacturing operations the desired response level may be artificially lowered in order to pre-calibrate the sensor. With such an artificially lowered desired level, for a pre-sensor that has a response within that desired artificially low level, a pre-determined portion of the pre-sensor's active area may be removed to obtain a sensor with the actual desired level; for a pre-sensor that has a response above the desired artificially low, a larger portion than the pre-determined portion of the pre-sensor's active area may be removed to obtain a sensor with the actual desired level; and for a pre-sensor that has a response below the desired artificially low level, a smaller portion than the pre-determined portion of the pre-sensor's active area is removed in order to obtain a sensor with the actual desired level. In other words, using such a methodology, all the pre-sensors would be physically altered to obtain sensors with the same desired predetermined calibration.

The pre-sensor is modified in order to obtain the desired pre-determined calibration, either by altering the volume of the sample chamber or by the electrode area in the sample chamber.

In some embodiments, there may be no actual pre-sensor that is subsequently modified to form the sensor, but rather, a pre-sensor is used as a template for one or more sensors (e.g., batch or lot of sensors, e.g., at least 100 sensors, at least 1,000 sensors, or even at least 50,000 sensors). For example, multiple sensors may be obtained from, e.g., a large sheet construction having working electrodes, counter electrodes and sample chambers. See for example, U.S. Pat. No. 6,338,790, particularly FIGS. 31A and 31B and the description associated therewith, which describes methods of making a plurality of sensors from a large sandwiched sheet construction. From this sheet, one (or more) test sensors could be removed (e.g., punched) using a standard template (e.g., shape and size), and these test sensors could be tested for their difference from the desired slope and y-intercept, and the results typically averaged. Subsequently removed sensors would be modified from the test sensor, as needed, by removing (e.g., punching) an appropriately shaped and sized sensor, which may differ from the test sensors, to obtain the desired slope and y-intercept. In this method, the test sensors provide a guide for the modification needed, so that each sensor is not individually tested.

Sensors may be available at pharmacies, hospitals, clinics, from doctors, and other sources of medical devices. Multiple sensors may be packaged together and sold as a single unit; e.g., a package of about 25, about 50, or about 100 sensors, or any other suitable number. A kit may include one or more sensors, and additional components such as control solutions and/or lancing device and/or meter, etc.

Sensors may be used for an electrochemical assay, or, for a photometric test. Sensors are generally configured for use with an electrical meter, which may be connectable to various electronics. A meter may be available at generally the same locations as the sensors, and sometimes may be packaged together with the sensors, e.g., as a kit.

Examples of suitable electronics connectable to the meter include a data processing terminal, such as a personal computer (PC), a portable computer such as a laptop or a handheld device (e.g., personal digital assistants (PDAs)), and the like. The electronics are configured for data communication with the receiver via a wired or a wireless connection. Additionally, the electronics may further be connected to a data network (not shown) for storing, retrieving and updating data corresponding to the detected glucose level of the user.

The various devices connected to the meter may wirelessly communicate with a server device, e.g., using a common standard such as 802.11 or Bluetooth RF protocol, or an IrDA infrared protocol. The server device could be another portable device, such as a Personal Digital Assistant (PDA) or notebook computer, or a larger device such as a desktop computer, appliance, etc. In some embodiments, the server device has a display, such as a liquid crystal display (LCD), as well as an input device, such as buttons, a keyboard, mouse or touch-screen. With such an arrangement, the user can control the meter indirectly by interacting with the user interface(s) of the server device, which in turn interacts with the meter across a wireless link.

The server device may also communicate with another device, such as for sending data from the meter and/or the service device to a data storage or computer. For example, the service device could send and/or receive instructions (e.g., an insulin pump protocol) from a health care provider computer. Examples of such communications include a PDA synching data with a personal computer (PC), a mobile phone communicating over a cellular network with a computer at the other end, or a household appliance communicating with a computer system at a physician's office.

A lancing device or other mechanism to obtain a sample of biological fluid, e.g., blood, from the patient or user may also be available at generally the same locations as the sensors and the meter, and sometimes may be packaged together with the sensor and/or meter, e.g., as a kit.

The sensors are particularly suited for inclusion in an integrated device, i.e., a device which has the sensor and a second element, such as a meter or a lancing device, in the device. The integrated device may be based on providing an electrochemical assay or a photometric assay. In some embodiments, sensors may be integrated with both a meter and a lancing device. Having multiple elements together in one device reduces the number of devices needed to obtain an analyte level and facilitates the sampling process. For example, embodiments may include a housing that includes one or more of the sensor strips, a skin piercing element and a processor for determining the concentration of an analyte in a sample applied to the strip. A plurality of sensors may be retained in a cassette in the housing interior and, upon actuation by a user, a single sensor may be dispensed from the cassette so that at least a portion extends out of the housing for use.

### Operation of the Sensor Strip

In use, a sample of biological fluid is provided into the sample chamber of the sensor, where the level of analyte is determined. The analysis may be based on providing an electrochemical assay or a photometric assay. In many embodiments, it is the level of glucose in blood that is determined. Also in many embodiments, the source of the biological fluid is a drop of blood drawn from a patient, e.g., after piercing the patient's skin with a lancing device, which could be present in an integrated device, together with the sensor strip.

Prior to providing the sample to the sensor, or even after providing the sample to the sensor, there is no need for the user to input a calibration code or other information regarding the operation and/or interaction of the sensor with the meter or other equipment. The sensor is configured so that the results received from the analysis are clinically accurate, without the user having to adjust the sensor or the meter. The sensor is physically configured to provide accurate results that are repeatable by a batch of sensors.

After receipt of the sample in the sensor, the analyte in the sample is, e.g., electrooxidized or electroreduced, at the working electrode and the level of current obtained at the counter electrode is correlated as analyte concentration. The sensor may be operated with or without applying a potential to the electrodes. In one embodiment, the electrochemical reaction occurs spontaneously and a potential need not be applied between the working electrode and the counter electrode. In another embodiment, a potential is applied between the working electrode and the counter electrode.

FIG. 10 shows another embodiment of a test strip sensor 910. Sensor 910 includes a rectangular wake-up bar 30 and is side-filled as indicated by opposing sample application areas 912.

FIG. 11 shows another embodiment of a test strip sensor 920. Sensor 920 includes a wavy-shaped wake-up bar 800. Wake-up bar 800 includes long sides that extend at either end parallel to the long axis of the strip 920. The two ends of wake-up bar 920 are offset in the direction of the short axis of the strip 920. A middle segment of wake-up bar 800 connects the offset end segments by extending at an angle to the long axis of the strip 920 so that corresponding corners of the end segments connect with the middle segment. Multiple middle sections may be provided, and the end segments may extend other than parallel to the long axis of strip 920, such as at any angle up to and including 90°. The wake-up bar 800 could be a simple rectangle or parallelogram, or may be trezoidally-shaped, or may have no parallel sides, and may have one or more curved sides or segments of sides, i.e., that are not straight.

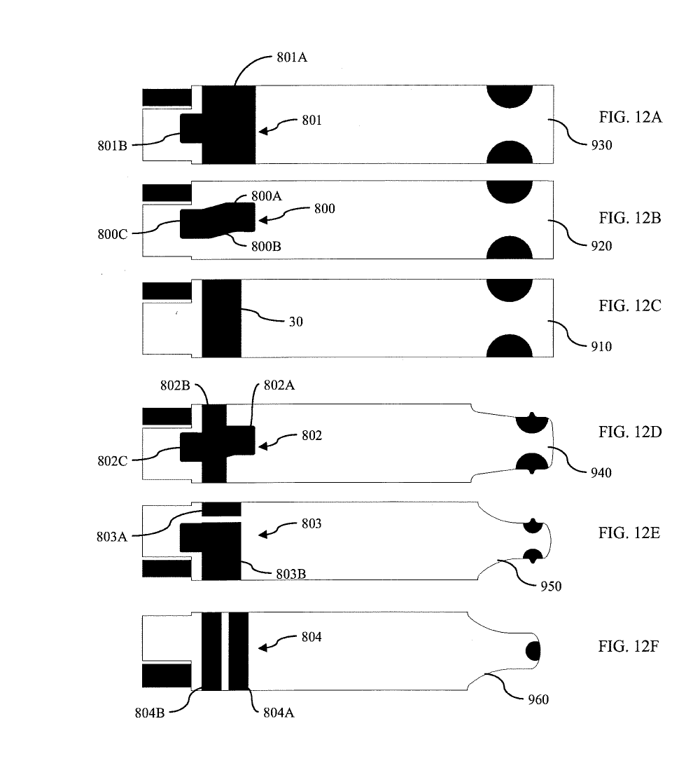

FIGS. 12A-12F shows embodiments of test strip sensors that have various wake-up bar shapes and various contours of their reagent ends or sample application ends. FIG. 12A illustrates a strip 930 having a wake-up bar 801 including a first rectangular section 801A and a second rectangular section 801B that are connected as sharing a common side. Section 801B may be a square, rhombus, parallelogram, or trapezoid, or may have a triangular shape. Sections 801A-B may be connected by a third or middle section. Any of the sides of sections 801A-B may be curved. Strip 930 is side-filled but may be end-filled (see, e.g., FIG. 12F).

FIGS. 12B and 12C include strips 920 and 910, respectively, of FIGS. 11 and 10. The wake-up bar 30 of FIG. 12C includes a single rectangle, while the wake-up bar 800 of strip 920 includes a rectangle 800A, which may be a square, a parallelepiped 800B, which may be a rhombus, and another rectangle 800C, which may be a square.

FIG. 12D includes an illustration of strip 940 which has a substantially cross-shaped wake-up bar 802. Wake-up bar 802 includes three sections 802A, 802B and 802C, which may each be rectangles of same or different dimensions. Section 802B extends across the short dimension of the strip from one side to the other, while sections 802A and 802C are shorter in the short dimension of the strip. Section 802A is also slightly longer than section 802C, although they may be the opposite or same length. One or both of sections 802A and 802C may be offset with respect to the long axis of the strip, and/or each other, as shown.

FIG. 12E illustrates another strip 950 having wake-up bar 803 which includes a L-shaped section 803B and a rectangular section 803A which are not connected on the top layer.

FIG. 12F illustrates another strip 960 having wake-up bar 804 including two rectangular sections 804A and 804B which are not connected on the top layer. For illustration, the strip 960 is end-filled, and any of the strips illustrated herein may be end-filled and/or side-filled on one or both sides, and/or even top or bottom filled, and may be provided in conjunction with any of the wake-up bars or reagent end contours illustrated or described herein or equivalents.

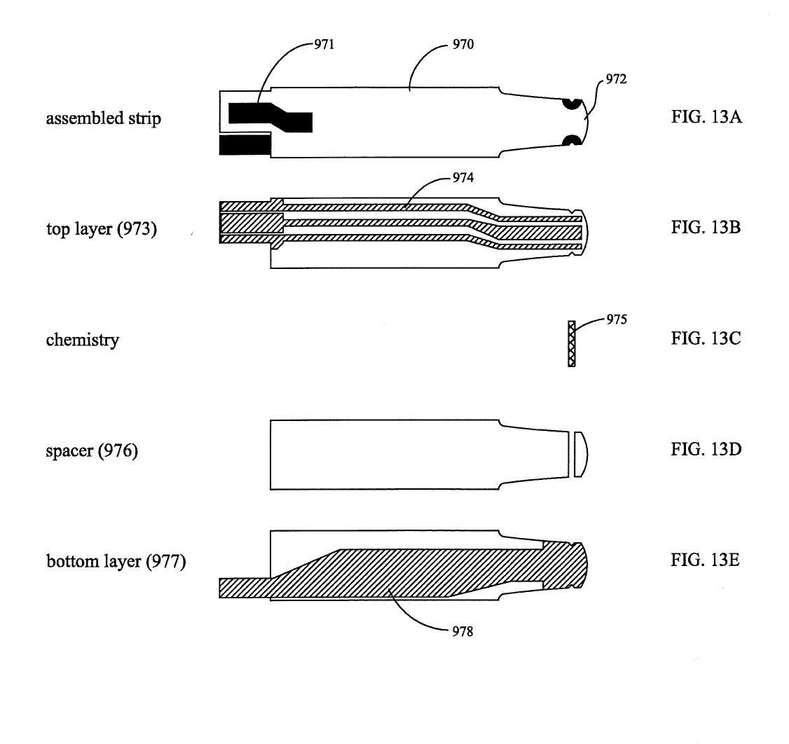

FIGS. 13A-13F illustrate various layers of an embodiment of a test strip having a distinctly contoured reagent end and wake-up bar shape. FIG. 13A illustrated a fully assembled strip 970 with exemplary wake-up bar 971 and side-fill reagent end 972. FIG. 13B illustrates a top layer 973 of strip 970, including conductive traces 974 corresponding to reference and indicator electrodes. FIG. 13C illustrates a chemistry section 975 of a reagent end 972 of strip 970. FIG. 13D illustrates a spacer 976 of strip 970 including an adhesive. FIG. 13E illustrates a bottom layer 977 of strip 970 including a conductive portion 978 corresponding to a working electrode.

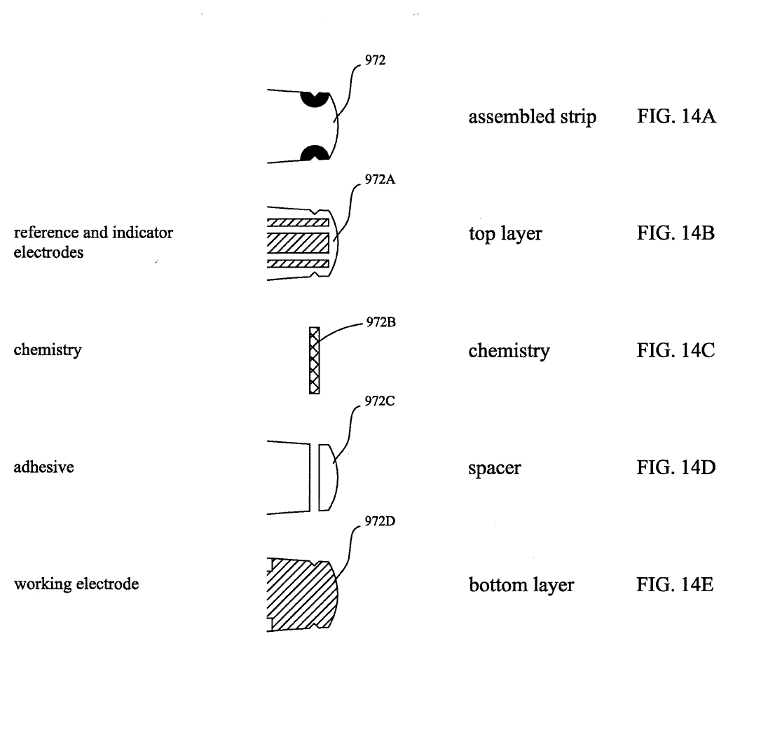

FIGS. 14A-14E illustrate various layers of the reagent end of the test strip illustrated at FIGS. 13A-13F. FIG. 14A illustrates a fully assembled reagent end 972 of test strip 970, while FIGS. 14B-13E illustrate top layer 972A, chemistry 972B, spacer 972C and bottom layer 972D, respectively, of reagent end 972 of strip 970.

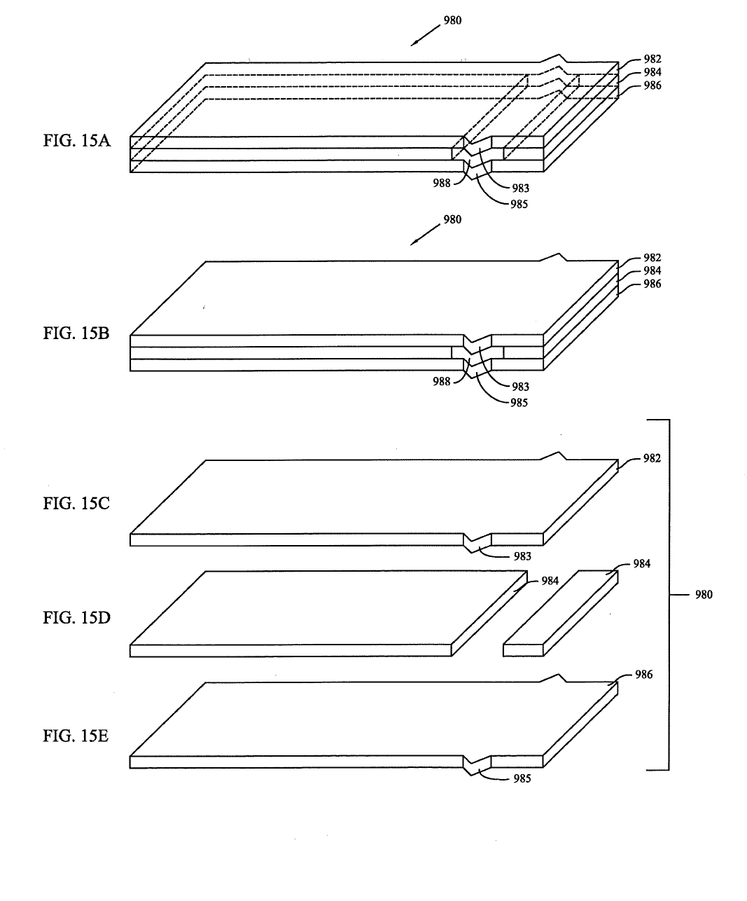

FIGS. 15A-15E illustrate embodiments of strips having distinct protrusions 983, 985 on opposing sides. FIGS. 15A and 15B show an assembled strip 980, while FIGS. 15C-E together illustrates an exploded view of strip 980. The strip 980 includes top layer 982, spacer 984 and bottom layer 986. Top layer 982 includes protrusion 983, while bottom layer 986 includes protrusion 985. Spacer 984 has a gap defining a sample acquisition channel 988 defined at a position corresponding to sections of top and bottom layers 982, 986 which include the protrusions 983, 985. Similar protrusions are provided at the opposite side of the strip 980. A user may apply a body fluid sample, e.g., a blood sample, into the channel 988 where the chemistry resides between electrodes for measuring an analyte such as glucose or lactate.

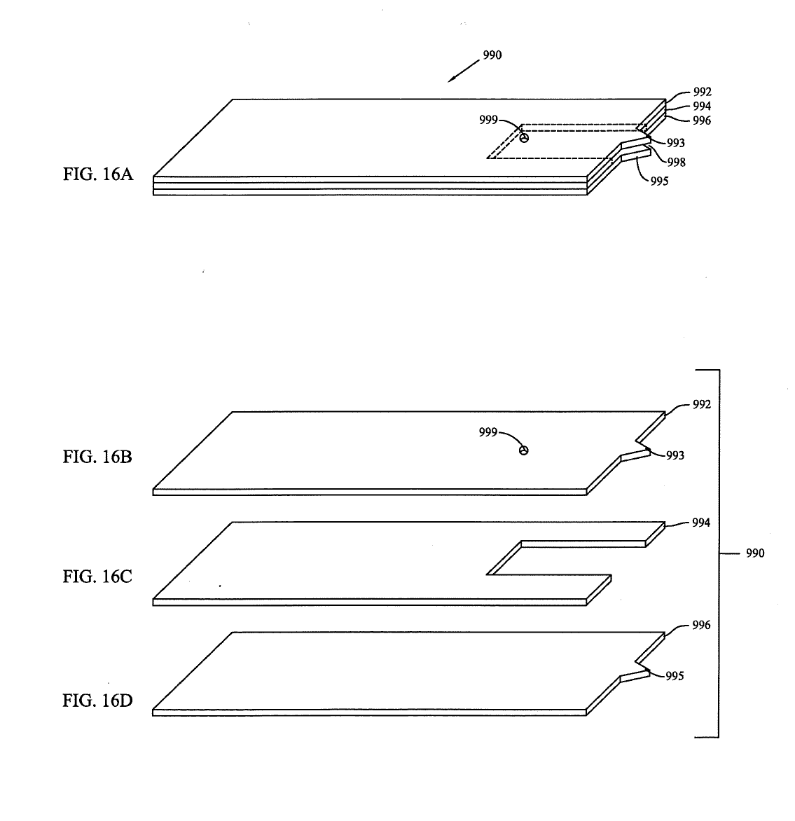

FIGS. 16A-16D illustrate embodiments of strips having a distinct protrusion at one end. FIG. 16A shows an assembled strip 990, while FIGS. 16B-D together illustrate an exploded view of strip 990. The strip includes top layer 992, spacer 994 and bottom layer 996. Top layer 992 includes protrusion 993, and bottom layer includes protrusion 995. Spacer 994 has a gap defining a sample acquisition channel 998 defined at a position corresponding to sections of top and bottom layers 992, 996 which include the protrusions 993, 995. A vent 999 is defined in top layer 992. A user may apply a body fluid sample, e.g., a blood sample, into the channel 998 where the chemistry resides between electrodes for measuring an analyte such as glucose or lactate.

Accordingly, a test strip in one embodiment, may include a reagent end for receiving a body fluid sample and an insertion end which is received at a test strip receptacle slot of a measuring device, the insertion end including one or more on-strip indicators for indicating to a measuring device that the test strip has been inserted into the device so that the device will power-up in preparation to perform a test, and the one or more on-strip indicators having a distinct pattern indicating whether calibration is automatic or whether the user needs to input calibration information to the device.

Accordingly, a test strip in one embodiment, may include a reagent end for receiving a body fluid sample and an insertion end which is received at a test strip receptacle slot of a measuring device, the insertion end including one or more of a distinct contour, perimeter pattern, shape, thickness, width or other geometric, material or other measurable property, or combinations thereof, indicating whether calibration is automatic or whether the user needs to input calibration information to the device.

Accordingly, a test strip in one embodiment, may include a reagent end for receiving a body fluid sample and an insertion end which is received at a test strip receptacle slot of a measuring device, the insertion end including one or more on-strip indicators for indicating to the device that the test strip has been inserted into the device so that the device will power-up in preparing to perform a test, and the one or more on-strip indicators having a distinct pattern indicating a predetermined calibration requirement corresponding to a type of the test strip which contrasts with different calibration requirements of at least one other type of test strip.

Accordingly, a test strip in one embodiment, may include a reagent end for receiving a body fluid sample and an insertion end which is received at a test strip receptacle slot of the measuring device, the insertion end including one or more of a distinct contour, perimeter pattern, shape, thickness, width or other geometric, material or other measurable property, or combinations thereof, indicating a particular calibration requirement corresponding to a type of the test strip which contrasts with different calibration requirements of at least one other type of test strip.

In another aspect, the user may enter a calibration information corresponding to a package of strips.

In another aspect, the user may enter a calibration code to the measuring device.

In another aspect, calibration may be automatic.

In another aspect, the test strip may be an electrochemical test strip.

In another aspect, the test strip may include a working electrode and a reference electrode in a coplanar arrangement.

In another aspect, the test strip may include a working electrode and a reference electrode in opposing arrangements at the reagent end.

In another aspect, the test strip may comprise a top fill, side fill or corner fill configuration, or combinations thereof.

In another aspect, the reagent end may comprise one or more of a rectangular or tapered shape, or a combination thereof.

In another aspect, the reagent end may include a sample fill area including one or more protrusions, cut-outs, notches, or indentations, or combinations thereof.

In another aspect, the strip may be configured for amperometric or coulometric measurements, or both.

Accordingly, a glucose meter device in one embodiment, may include a test strip receptacle slot that is configured for receiving a glucose monitoring test strip which includes a reagent end for receiving a body fluid sample and an insertion end which is received at the glucose meter test strip receptacle slot, and the insertion end including one or more wake-up bars for indicating to the glucose meter that the test strip has been inserted into the meter so that the meter will power-up in preparation to perform a test, and the one or more wake-up bars have a distinct pattern that is identifiable by the meter device indicating whether calibration is automatic or whether the user needs to input calibration information to the meter.

Accordingly, a glucose meter device in one embodiment, may include a test strip receptacle slot that is configured for receiving a glucose monitoring test strip which includes a reagent end for receiving a body fluid sample and an insertion end which is received at the test strip receptacle slot, the insertion end including one or more of a distinct contour, perimeter pattern, shape, thickness, width or other geometric, material or other measurable property, or combinations thereof, that is identifiable by the meter device indicating whether calibration is automatic or whether the user needs to input calibration information to the meter.

Accordingly, a glucose meter device in one embodiment, may include a test strip receptacle slot that is configured for receiving a glucose monitoring test strip which includes a reagent end for receiving a body fluid sample and an insertion end which is received at a glucose meter test strip receptacle slot, the insertion end including one or more wake-up bars for indicating to a glucose meter that the test strip has been inserted into the meter so that the meter will power-up in preparing to perform a test, and the one or more wake-up bars have a distinct pattern that is identifiable by the meter device indicating a particular calibration requirement corresponding to a type of the test strip which contrasts with different calibration requirements of at least one other type of test strip.

Accordingly, a glucose meter device in one embodiment, may include a test strip receptacle slot that is configured for receiving a glucose monitoring test strip which includes a reagent end for receiving a body fluid sample and an insertion end which is received at the test strip receptacle slot, the insertion end including one or more of a distinct contour, perimeter pattern, shape, thickness, width or other geometric, material or other measurable property, or combinations thereof, that is identifiable by the meter device indicating a calibration requirement corresponding to a type of the test strip which contrasts with different calibration requirements of at least one other type of test strip.

In another aspect, the user may enter a calibration code corresponding to a package of strips.

In another aspect, the user may insert a predetermined test strip that includes a memory chip that has calibration information on it for transmitting to the meter.

In another aspect, calibration of a meter may be automatic.

In another aspect, the meter may include a program code embodied within a processor-readable medium for identifying a type of test strip inserted at the test strip receptacle slot and proceeding with a glucose test procedure in accordance with a predetermined calibration requirement corresponding to that type of strip.

In another aspect, the test strip receptacle of a meter may be configured to identify the insertion end of the test strip inserted at the test strip receptacle.

In another aspect, the test strip receptacle of a meter may be configured to identify the test strip inserted at the test strip receptacle.

In another aspect, the strip may be configured for amperometric or coulometric measurements, or both.

The invention has been described with reference to various specific and preferred embodiments and techniques. However, it will be apparent to one of ordinarily skill in the art that many variations and modifications may be made while remaining within the spirit and scope of the invention.

All patents and other references in this specification are indicative of the level of ordinary skill in the art to which this invention pertains. All patents are herein incorporated by reference to the same extent as if each individual patent was specifically and individually incorporated by reference.

1.-21. (canceled)

22. An analyte meter device, comprising:
a test strip receptacle slot configured to receive a first, second and third type of test strip, each type comprising a reagent end for receiving a sample, wherein the analyte meter device is configured to identify
(i) the first type of test strip, for which calibration information is automatically communicated to the meter,
(ii) the second type of test strip, which requires that calibration information for the test strip be input by a user of the analyte meter, and
(iii) the third type of test strip, for which calibration information is not automatically communicated to the meter and which does not require calibration information to be inputted by the user; and
a processor, wherein the processor is configured to receive from the first, second and third types of test strips one or more signals indicative of an analyte concentration in the sample and thereby determine the analyte concentration in the sample.

23. The analyte meter device of claim 22, wherein the analyte meter device identifies the first, second and third types of test strips mechanically.

24. The analyte meter device of claim 22, wherein the analyte meter device identifies the first, second and third types of test strips electrically.

25. The analyte meter device of claim 22, wherein the analyte is glucose.

26. The analyte meter device of claim 22, further comprising a display configured to display the one or more signals and/or the analyte concentration in the sample.

27. The analyte meter device of claim 26, wherein the display is further configured to display a rate of change of the analyte concentration.

28. The analyte meter device of claim 26, wherein the display is further configured to indicate exceeding of a threshold analyte concentration.

29. The analyte meter device of claim 28, wherein the exceeding of a threshold analyte concentration is indicative of hyper-glycemia or hypo-glycemia.

30. The analyte meter device of claim 22, further comprising a port configured for coupling to a display.

31. The analyte meter device of claim 22, wherein the analyte meter device is configured for data communication with a personal computer and/or a network.

32. The analyte meter device of claim 31, wherein the analyte is glucose and the data corresponds to the glucose level of a user of the analyte meter device.

33. The analyte meter device of claim 31, wherein the data communication with the personal computer and/or the network is wireless data communication.

34. The analyte meter device of claim 22, further comprising electronics coupled to the analyte meter device, wherein the electronics provide for wired or wireless data communication with a server device.

35. The analyte meter device of claim 34, wherein the server device is a personal digital assistant (PDA) and/or mobile phone, a notebook computer, or a desktop computer.

36. The analyte meter device of claim 34, wherein the server device is configured to send and/or receive instructions from a health-care provider computer.

37. The analyte meter device of claim 22, further comprising electronics coupled to the analyte meter device, wherein the electronics provide for wireless data communication with a server device.

38. The analyte meter device of claim 37, wherein the wireless data communication utilizes an 802.11 protocol, a Bluetooth RF protocol, or an IrDA infrared protocol.

39. A system comprising:
an analyte meter device, the analyte meter device comprising
a test strip receptacle slot configured to receive a first, second and third type of test strip, each type comprising a reagent end for receiving a sample, wherein the analyte meter device is configured to identify
(i) the first type of test strip, for which calibration information is automatically communicated to the meter,
(ii) the second type of test strip, which requires that calibration information for the test strip be input by a user of the analyte meter, and
(iii) the third type of test strip, for which calibration information is not automatically communicated to the meter and which does not require calibration information to be inputted by the user, and
a processor, wherein the processor is configured to receive from the first, second and third types of test strips one or more signals indicative of an analyte concentration in the sample and thereby determine the analyte concentration in the sample; and
a server device configured for wired or wireless data communication with the analyte meter device.

40. The system of claim 39, wherein the analyte meter device identifies the first, second and third types of test strips mechanically.

41. The system of claim 39, wherein the analyte meter device identifies the first, second and third types of test strips electrically.

42. The system of claim 39, wherein the analyte is glucose.

43. The system of claim 39, wherein the analyte meter device further comprises a display configured to display the one or more signals and/or the analyte concentration in the sample.

44. The system of claim 43, wherein the display is further configured to display a rate of change of the analyte concentration.

45. The system of claim 43, wherein the display is further configured to indicate exceeding of a threshold analyte concentration.

46. The system of claim 45, wherein the exceeding of a threshold analyte concentration is indicative of hyper-glycemia or hypo-glycemia.

47. The system of claim 39, wherein the analyte meter device further comprises a port configured for coupling to a display.

48. The system of claim 39, wherein the analyte is glucose and the data corresponds to the glucose level of a user of the analyte meter device.

49. The system of claim 39, wherein the server device is configured for wireless data communication with the analyte meter device.

50. The system of claim 49, wherein the wireless data communication utilizes an 802.11 protocol, a Bluetooth RF protocol, or an IrDA infrared protocol.

51. The system of claim 39, wherein the server device is a personal digital assistant (PDA) and/or mobile phone, a notebook computer, or a desktop computer.

52. The system of claim 39, wherein the server device is configured to send and/or receive instructions from a health-care provider computer.

53. The system of claim 39, wherein the server device comprises a display.

54. The system of claim 53, wherein the display is a liquid crystal display (LCD).

55. The system of claim 39, wherein the server device comprises a touchscreen.

56. An glucose meter device, comprising:
a test strip receptacle slot configured to receive a first, second and third type of glucose test strip, each type comprising a reagent end for receiving a sample, wherein the glucose meter device is configured to identify
(i) the first type of test strip, for which calibration information is automatically communicated to the meter,
(ii) the second type of test strip, which requires that calibration information for the test strip be input by a user of the analyte meter, and
(iii) the third type of test strip, for which calibration information is not automatically communicated to the meter and which does not require calibration information to be inputted by the user, and
a processor, wherein the processor is configured to receive from the first, second and third types of test strips one or more signals indicative of a glucose concentration in the sample and thereby determine the glucose concentration in the sample; and
electronics coupled to the glucose meter device, wherein the electronics provide for wireless data communication with a server device.

57. The glucose meter device of claim 56, wherein the analyte meter device identifies the first, second and third types of test strips mechanically.

58. The glucose meter device of claim 56, wherein the analyte meter device identifies the first, second and third types of test strips electrically.

59. The glucose meter device of claim 56, further comprising a display configured to display the one or more signals and/or the glucose concentration in the sample.

60. The glucose meter device of claim 59, wherein the display is further configured to display a rate of change of the glucose concentration.

61. The glucose meter device of claim 59, wherein the display is further configured to indicate the exceeding of a threshold glucose concentration.

62. The glucose meter device of claim 61, wherein the exceeding of a threshold glucose concentration is indicative of hyper-glycemia or hypo-glycemia.

63. The glucose meter device of claim 56, further comprising a port configured for coupling to a display.

64. The glucose meter device of claim 56, wherein the glucose meter device is configured for data communication with a personal computer and/or a network.

65. The glucose meter device of claim 64, wherein the data communication with the personal computer and/or the network comprises the glucose level of a user of the glucose meter device.

66. The glucose meter device of claim 64, wherein the data communication with the personal computer and/or the network is wireless data communication.

67. The glucose meter device of claim 56, wherein the server device is a personal digital assistant (PDA) and/or mobile phone, a notebook computer, or a desktop computer.

68. The glucose meter device of claim 56, wherein the server device is configured to send and/or receive instructions from a health-care provider computer.

69. The glucose meter device of claim 56, wherein the wireless data communication utilizes an 802.11 protocol, a Bluetooth RF protocol, or an IrDA infrared protocol.
